(19) United States
(12) Patent Application Publication
(10) Pub. No.: US 2025/0101020 A1
(43) Pub. Date: Mar. 27, 2025

(54) SPIROCYCLIC LACTAM INHIBITORS

(71) Applicant: Incyte Corporation, Wilmington, DE (US)

(72) Inventors: Stacey Shepard, Wilmington, DE (US); Lixin Shao, Wilmington, DE (US); Xiaozhao Wang, Moorestown, NJ (US); Eddy W. Yue, Landenberg, PA (US)

(21) Appl. No.: 18/891,232

(22) Filed: Sep. 20, 2024

Related U.S. Application Data
(60) Provisional application No. 63/539,688, filed on Sep. 21, 2023.

(51) Int. Cl. C07D 471/04; A61K 31/437; A61K 31/4709; C07D 519/00

(52) U.S. Cl. CPC C07D 471/04; A61K 31/437; A61K 31/4709; C07D 519/00

(57) ABSTRACT

The present application provides bicyclic spriolactam compounds that modulate the activity of JAK2, which are useful in the treatment of various diseases, including cancer.

### TECHNICAL FIELD

The present invention provides bicyclic spriolactam compounds that modulate the activity of JAK2 and are useful in the treatment of diseases related to JAK2, including cancer.

### BACKGROUND

Janus kinase (JAK) 2 plays pivotal roles in signaling by several cytokine receptors. The mutant JAK2 V617F is the most common molecular event associated with myeloproliferative neoplasms. Selective targeting of the JAK2 V617F mutant may be useful for treating various pathologies, while sparing essential JAK2 functions. This application is directed to this need and others.

### SUMMARY

The present invention relates to, inter alia, compounds of Formula I:

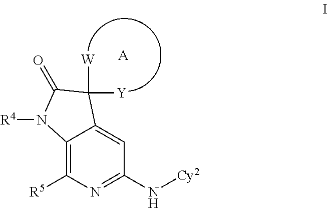

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

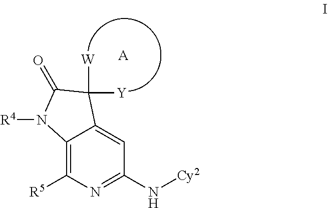

or a pharmaceutically acceptable salt thereof, wherein:
W is CR^{W} or C(R^{W})₂;
Y is CR^{Y} or C(R^{Y})₂;
each R^{W} is independently selected from H, oxo, C₁₋₆ alkyl, C₁₋₆ haloalkyl, C₂₋₆ alkenyl, and C₂₋₆ alkynyl;
each R^{Y} is independently selected from H, oxo, C₁₋₆ alkyl, C₁₋₆ haloalkyl, C₂₋₆ alkenyl, and C₂₋₆ alkynyl;

Ring A is selected from C₃₋₁₀ cycloalkyl and 4-10 membered heterocycloalkyl, wherein the C₃₋₁₀ cycloalkyl and 4-10 membered heterocycloalkyl of Ring A are each optionally substituted with 1, 2, 3, 4, 5, 6, 7, or 8 independently selected R¹ substituents;
each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a1}, SR^{a1}, NHOR^{a1}, C(O)R^{b1}, C(O)NR^{c1}R^{d1}, C(O)NR^{c1} (OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1}) R^{b1}, C(═NR^{e1}) NR^{c1}R^{d1}, NR^{c1}C(═NR^{e1}) NR^{c1}R^{d1}, NR^{c1}C(═NR^{e1}) R^{b1}, NR^{c1}S(O)R^{b1}, NR^{c1}S(O)NR^{c1}R^{d1}, NR^{c1}S(O)₂R^{b1}, NR^{c1}S(O)(═NR^{e1}) R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1}) R^{b1}, and OS(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{c1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁-6 alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{c1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11}) R^{b11}, C(═NR^{e11}) NR^{c11}R^{d11}, NR^{c11}C(═NR^{e11}) NR^{c11}R^{d11}, NR^{c11}C(═NR^{e11}) R^{b11}, NR^{c11}S(O)R^{b11}, NR^{c11}S(O)NR^{c11}R^{d11}, NR^{c11}S(O)₂R^{b11}, NR^{c11}S(O)(═NR^{e11}) R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11}) R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of RIA are each optionally substituted with 1, 2, 3, 4, 5, 6, 7, or 8 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R^{1B} substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1B} substituents;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b11} are each optionally substituted with 1, 2, 3, 4, 5, 6, 7, or 8 independently selected R^{1B} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, SR^{a12}, NHOR^{a12}, C(O)R^{b12}, C(O)NR^{c12}R^{d12}, C(O)NR^{c12} (OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12}) R^{b12}, C(═NR^{e12}) NR^{c12}R^{d12}, NR^{c12}C(═NR^{e12}) NR^{c12}R^{d12}, NR^{c12}C(═NR^{e12}) R^{b12}, NR^{c12}S(O)R^{b12}, NR^{c12}S(O)NR^{c12}R^{d12}, NR^{c12}S(O)₂R^{b12}, NR^{c12}S(O)(═NR^{e12}) R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12}) R^{b12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl- and (4-7 membered heterocycloalkyl)-C₁₋₆ alkyl- of R^{1B} are each optionally substituted with 1, 2, 3, or 4 independently selected R^{1C} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a12}, R^{c12} and R^{d12} are each optionally substituted with 1, 2, 3, or 4 independently selected R^{1C} substituents;
or, any R^{c12} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{1C} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a13} SR^{a13}, NHOR^{a13}, C(O)R^{b13}, C(O)NR^{c13}R^{d13}, C(O)NR^{c13}(OR^{a13}), C(O)OR^{a13}, OC(O)R^{b13} OC(O)NR^{c13}R^{d13}, NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13}) R^{b13}, C(═NR^{e13}) NR^{c13}R^{d13}, NR^{c13}C(═NR^{e13}) NR^{c13}R^{d13} NR^{c13}C(═NR^{e13}) R^{b13}, NR^{c13}S(O)R^{b13}, NR^{e13}S(O)NR^{c13}R^{d13}, NR^{c13}S(O)₂R^{b13} NR^{c13}S(O)(═NR^{e13}) R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13}) R^{b13}, and OS(O)₂R^{b13}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl- and (4-7 membered heterocycloalkyl)-C₁₋₆ alkyl- of R^{1C} are each optionally substituted with 1, 2, 3, or 4 independently selected R^{M} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a13}, R^{c13} and R^{d13} are each optionally substituted with 1, 2, 3, or 4 independently selected R^{M} substituents;
or, any R^{c13} and R^{d13} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b13} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b13} are each optionally substituted with 1, 2, 3, or 4 independently selected R^{M} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;

Cy² is selected from C₆₋₁₀ aryl, C₃₋₁₀ cycloalkyl, 5-10 membered heteroaryl, and 4-10 membered heterocycloalkyl, wherein the C₆₋₁₀ aryl, C₃₋₁₀ cycloalkyl, 5-10 membered heteroaryl, and 4-10 membered heterocycloalkyl of Cy² are each optionally substituted with 1, 2, 3, 4, 5, 6, 7, or 8 independently selected R² substituents;
each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2}) NR^{c2}R^{d2}, NR^{c2}C(═NR^{c2}) NR^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, and OS(O)₂R^{b2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁-6 alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21} (OR^{a21}), C(O)OR^{a21}, OC(O)R^{b21} OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21}) NR^{c21}R^{d21}, NR^{c21}C(═NR^{e21}) NR^{c21}R^{d21} NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{e21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22} (OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22}) R^{b22}, C(═NR^{e22}) NR^{c22}R^{d22} NR^{c22}C(═NR^{e22}) NR^{c22}R^{d22}, NR^{c22}C(═NR^{e22}) R^{b22}, NR^{c22}S(O)R^{b22}, NR^{c22}S(O)NR^{c22}R^{d22}, NR^{c22}S(O)₂R^{b22}, NR^{c22}S(O)(═NR^{e22}) R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22}) R^{b22}, and OS(O)₂R^{b22}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2B} are each optionally substituted with 1, 2, 3, 4, 5, 6, 7, or 8 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a22}, R^{c22} and R^{d22} are each optionally substituted with 1, 2, 3, 4, 5, 6, 7, or 8 independently selected R^{2C} substituents;
or, any R^{c22} and R^{d22} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2C} substituents;
each R^{b22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b22} are each optionally substituted with 1, 2, 3, 4, 5, 6, 7, or 8 independently selected R^{2C} substituents;
each R^{c22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a23} SR^{a23}, NHOR^{a23}, C(O)R^{b23}, C(O)NR^{c23}R^{d23}, C(O)NR^{c23} (OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR^{c23}R^{d23}, C(═NR^{e23}) R^{b23}, C(═NR^{e23}) NR^{c23}R^{d23}, NR^{c23}C(═NR^{e23}) NR^{c23}R^{d23} NR^{c23}C(═NR^{e23}) R^{b23}, NR^{c23}S(O)R^{b23}, NR^{c23}S(O)NR^{c23}R^{d23}, NR^{c23}S(O)₂R^{b23} NR^{c23}S(O)(═NR^{e23}) R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, OS(O)(═NR^{e23}) R^{b23}, and OS(O)₂R^{b23}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a23}, R^{e23}, and R^{d23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a23}, R^{c23} and R^{d23} are each optionally substituted with 1, 2, 3, or 4 independently selected R^{M} substituents;
or, any R^{c23} and R^{d23} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b23} are each optionally substituted with 1, 2, 3, or 4 independently selected R^{M} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a5}, NH₂, and NHC₁₋₆ alkyl, wherein the C₁₋₆ alkyl is optionally substituted with OH, CN, and NH₂;
R^{a5} is H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂; and each R^{M} is independently selected from H, OH, halo, oxo, CN, C(O)OH, C(O)NH₂, C(O)NH(C₁₋₄ alkyl), C(O)N(C₁₋₆ alkyl)₂, NH₂, NO₂, SF5, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-.

In some embodiments, W is C(R^{W})₂.

In some embodiments, W is CR^{W}.

In some embodiments, each R^{W} is independently selected from H, oxo, and C₁₋₆ alkyl.

In some embodiments, each R^{W} is independently selected from H and oxo.

In some embodiments, each R^{W} is H.

In some embodiments, W is CH₂.

In some embodiments, Y is C(R^{Y})₂.

In some embodiments, Y is CR^{Y}.

In some embodiments, each R^{Y} is independently selected from H, oxo, and C₁₋₆ alkyl.

In some embodiments, each R^{Y} is independently selected from H and oxo.

In some embodiments, each R^{Y} is H.

In some embodiments, Y is CH₂.

In some embodiments, W is C(R^{W}) 2 and Y is C(R^{Y})₂.

In some embodiments, W is CH₂ and Y is CH₂.

In some embodiments, Ring A is C₃₋₁₀ cycloalkyl, wherein the C₃₋₁₀ cycloalkyl of Ring A is optionally substituted with 1, 2, 3, 4, 5, 6, 7, or 8 independently selected R¹ substituents.

In some embodiments, Ring A is C₃₋₁₀ cycloalkyl, wherein the C₃₋₁₀ cycloalkyl of Ring A is optionally substituted with 1, 2, 3, or 4 independently selected R¹ substituents.

In some embodiments, Ring A is C₃₋₇ cycloalkyl, wherein the C₃₋₇ cycloalkyl of Ring A is optionally substituted with 1, 2, 3, or 4 independently selected R¹ substituents.

In some embodiments, Ring A is selected from cyclobutyl and cyclohexyl, wherein the cyclobutyl and cyclohexyl of Ring A are each optionally substituted with 1, 2, 3, or 4 independently selected R¹ substituents.

In some embodiments, Ring A is cyclobutyl, which is optionally substituted with 1, 2, 3, or 4 independently selected R¹ substituents.

In some embodiments, Ring A is cyclohexyl, which is optionally substituted with 1, 2, 3, or 4 independently selected R¹ substituents.

In some embodiments, each R¹ is independently selected from C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, NR^{c1}C(O)R^{b1}, NR^{c1}C(O)OR^{a1}, and NR^{c1}C(O)NR^{c1}R^{d1}, wherein the C₁₋₆ alkyl, C₂₋₆ alkenyl, and C₂₋₆ alkynyl of R¹ are each optionally substituted with 1, 2, 3, or 4 independently selected R^{1A} substituents.

In some embodiments, each R¹ is independently selected from C₁₋₆ alkyl, C₁₋₆ haloalkyl, NR^{c1}C(O)R^{b1}, NR^{c1}C(O)OR^{a1}, and NR^{c1}C(O)NR^{c1}R^{d1}, wherein each C₁₋₆ alkyl of R¹ is optionally substituted with 1, 2, 3, or 4 independently selected R^{1A} substituents.

In some embodiments, each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents.

In some embodiments, each R¹ is independently selected from C₁₋₃ alkyl and NR^{c1}C(O)NR^{c1}R^{d1}, wherein each C₁₋₃ alkyl of R¹ is optionally substituted with 1, 2, 3, or 4 independently selected R^{1A} substituents.

In some embodiments, each R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl.

In some embodiments, each R^{a1}, R^{b1}, R^{c1}, and R^{d1} is independently selected from H, C₁₋₆ alkyl, and C₁₋₆ haloalkyl.

In some embodiments, each R^{a1}, R^{b1}, R^{c1}, and R^{d1} is independently selected from H and C₁₋₆ alkyl.

In some embodiments, each R^{a1}, R^{b1}, R^{c1}, and R^{d1} is independently selected from H and C₁₋₃ alkyl.

In some embodiments, each R^{c1} and R^{d1} is independently selected from H, C₁₋₆ alkyl, C₁₋₆ haloalkyl, C₂₋₆ alkenyl, and C₂₋₆ alkynyl.

In some embodiments, each R^{c1} and R^{d1} is independently selected from H, C₁₋₆ alkyl, and C₁₋₆ haloalkyl.

In some embodiments, each R^{c1} and R^{d1} is independently selected from H and C₁₋₆ alkyl.

In some embodiments, each R^{c1} and R^{d1} is independently selected from H and C₁₋₃ alkyl.

In some embodiments, each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents; and each R^{c1} and R^{d1} is independently selected from H, C₁₋₆ alkyl, C₁₋₆ haloalkyl, C₂₋₆ alkenyl, and C₂₋₆ alkynyl.

In some embodiments, each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents; and each R^{c1} and R^{d1} is independently selected from H and C₁₋₆ alkyl.

In some embodiments, each R¹ is independently selected from C₁₋₃ alkyl and NR^{c1}C(O)NR^{c1}R^{d1}, wherein each C₁₋₃ alkyl of R¹ is optionally substituted with 1, 2, 3, or 4 independently selected R^{1A} substituents; and each R^{c1} and R^{d1} is independently selected from H and C₁₋₆ alkyl.

In some embodiments, each R¹ is independently selected from C₁₋₃ alkyl and NR^{c1}C(O)NR^{c1}R^{d1}, wherein each C₁₋₃ alkyl of R¹ is optionally substituted with 1, 2, 3, or 4 independently selected R^{1A} substituents; and each R^{c1} and R^{d1} is independently selected from H and C₁₋₃ alkyl.

In some embodiments, each R¹ is independently selected methyl and —NHC(O)NHCH₂CH₂, wherein each methyl of R¹ is optionally substituted with 1, 2, 3, or 4 independently selected R^{1A} substituents.

In some embodiments, each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11} NR^{c11}C(O)R^{b11}, NR^{c11}C(O)OR^{a11}, and NR^{c11}C(O)NR^{c11}R^{d11}.

In some embodiments, each R^{1A} is independently selected from OR^{a11}, NR^{c11}C(O)R^{b11}, and NR^{c11}C(O)OR^{a11}.

In some embodiments, each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl.

In some embodiments, each R^{a11}, R^{b11}, and R^{c11} is independently selected from H, C₁₋₆ alkyl, and C₃₋₁₀ cycloalkyl.

In some embodiments, each R^{a11}, R^{b11}, and R^{d11} is independently selected from H, C₁₋₆ alkyl, and C₃₋₇ cycloalkyl.

In some embodiments, each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}C(O)R^{b11}, NR^{c11}C(O)OR^{a11}, and NR^{c11}C(O)NR^{c11}R^{d11}, and each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}C(O)R^{b11}, NR^{c11}C(O)OR^{a11}, and NR^{c11}C(O)NR^{c11}R^{d11}; and each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl.

In some embodiments, each R^{1A} is independently selected from OR^{a11}, NR^{c11}C(O)R^{b11}, and NR^{c11}C(O)OR^{a11}; and each R^{a11}, R^{b11}, and R^{d11} is independently selected from H, C₁₋₆ alkyl, and C₃₋₇ cycloalkyl.

In some embodiments, each R^{1A} is independently selected OH, —NHC(O)OCH₃, and —NHC(O) (cyclopropyl).

In some embodiments, R⁴ is selected from H, C₁₋₆ alkyl, C₁₋₆ alkoxy, C₁₋₆ haloalkyl, and C₁₋₆ haloalkoxy.

In some embodiments, R⁴ is selected from H and C₁₋₆ alkyl.

In some embodiments, R⁴ is selected from H and C₁₋₃ alkyl.

In some embodiments, R⁴ is C₁₋₆ alkyl.

In some embodiments, R⁴ is C₁₋₃ alkyl.

In some embodiments, R⁴ is trideuteromethyl.

In some embodiments, R⁵ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

In some embodiments, R⁵ is selected from H and C₁₋₆ alkyl.

In some embodiments, R⁵ is selected from H and C₁₋₃ alkyl.

In some embodiments, R⁵ is H.

In some embodiments, Cy² is 5-10 membered heteroaryl, wherein the 5-10 membered heteroaryl of Cy² is optionally substituted with 1, 2, 3, 4, 5, 6, 7, or 8 independently selected R² substituents.

In some embodiments, Cy² is 5-10 membered heteroaryl, wherein the 5-10 membered heteroaryl of Cy² is optionally substituted with 1, 2, 3, or 4 independently selected R² substituents.

In some embodiments, Cy² is 8-10 membered heteroaryl, wherein the 8-10 membered heteroaryl of Cy² is optionally substituted with 1, 2, 3, or 4 independently selected R² substituents.

In some embodiments, Cy² is bicyclic 8-10 membered heteroaryl, wherein the bicyclic 8-10 membered heteroaryl of Cy² is optionally substituted with 1, 2, 3, or 4 independently selected R² substituents.

In some embodiments, Cy² is selected from quinolinyl and pyrazolo[4,3-b]pyridinyl, wherein the quinolinyl and pyrazolo[4,3-b]pyridinyl of Cy² are each optionally substituted with 1, 2, 3, or 4 independently selected R² substituents.

In some embodiments, Cy² is quinolinyl, which is optionally substituted with 1, 2, 3, or 4 independently selected R² substituents.

In some embodiments, Cy² is pyrazolo[4,3-b]pyridinyl, which is optionally substituted with 1, 2, 3, or 4 independently selected R² substituents.

In some embodiments, each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, 4, 5, 6, 7, or 8 independently selected R^{2A} substituents.

In some embodiments, each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents.

In some embodiments, each R² is independently selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents.

In some embodiments, each R² is independently selected from C₁₋₆ alkyl, C₁₋₆ haloalkyl and 5-10 membered heteroaryl, wherein each C₁₋₆ alkyl and 5-10 membered heteroaryl of R² is optionally substituted with 1, 2, 3, or 4 independently selected R^{2A} substituents.

In some embodiments, each R² is independently selected from C₁₋₆ alkyl, C₁₋₆ haloalkyl, and 5-6 membered heteroaryl, wherein each C₁₋₆ alkyl and 5-6 membered heteroaryl of R² is optionally substituted with 1, 2, 3, or 4 independently selected R^{2A} substituents.

In some embodiments, each R² is independently selected from C₁₋₃ alkyl, C₁₋₃ haloalkyl, and 5-10 membered heteroaryl, wherein each C₁₋₃ alkyl and 5-10 membered heteroaryl of R² is optionally substituted with 1, 2, 3, or 4 independently selected R^{2A} substituents.

In some embodiments, each R² is independently selected from C₁₋₃ alkyl, C₁₋₃ haloalkyl and 5-6 membered heteroaryl, wherein each C₁₋₃ alkyl and 5-6 membered heteroaryl of R² is optionally substituted with 1, 2, 3, or 4 independently selected R^{2A} substituents.

In some embodiments, each R² is independently selected from C₁₋₃ alkyl, C₁₋₃ haloalkyl and 5-6 membered heteroaryl, wherein each 5-6 membered heteroaryl of R² is optionally substituted with 1, 2, 3, or 4 independently selected R^{2A} substituents.

In some embodiments, each R² is independently selected from methyl, trifluoromethyl, and pyrazolyl, wherein each pyrazolyl of R² is optionally substituted with 1, 2, 3, or 4 independently selected R^{2A} substituents.

In some embodiments, each R^{2A} is independently selected from halo, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂-6 alkynyl of R^{2A} are each optionally substituted with 1, 2, 3, 4, 5, 6, 7, or 8 independently selected R^{2B} substituents.

In some embodiments, each R^{2A} is independently selected from halo, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂-6 alkynyl of R^{2A} are each optionally substituted with 1, 2, 3, or 4 independently selected R^{2B} substituents.

In some embodiments, each R^{2A} is independently selected from halo, C₁₋₆ alkyl, C₁₋₆ haloalkyl, C₂₋₆ alkenyl, and C₂₋₆ alkynyl.

In some embodiments, each R^{2A} is independently selected from C₁₋₆ alkyl and C₁₋₆ haloalkyl.

In some embodiments, each R^{2A} is independently selected from C₁₋₆ haloalkyl.

In some embodiments, each R^{2A} is independently selected from C₁₋₃ haloalkyl.

In some embodiments, each R^{2A} is trifluoromethyl.

In some embodiments:
W is CR^{W} or C(R^{W})₂;
Y is CR^{Y} or C(R^{Y})₂;
each R^{W} is independently selected from H, oxo, and C₁₋₆ alkyl;
each R^{Y} is independently selected from H, oxo, and C₁₋₆ alkyl;
Ring A is C₃₋₁₀ cycloalkyl, wherein the C₃₋₁₀ cycloalkyl of Ring A is optionally substituted with 1, 2, 3, or 4 independently selected R¹ substituents;
each R¹ is independently selected from C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, NR^{c1}C(O)R^{b1}, NR^{c1}C(O)OR^{a1}, and NR^{c1}C(O)NR^{c1}R^{d1}, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}C(O)R^{b11}, NR^{c11}C(O)OR^{a11}, and NR^{c11}C(O)NR^{c11}R^{d11};
each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl; Cy² is 5-10 membered heteroaryl, wherein the 5-10 membered heteroaryl of Cy² is optionally substituted with 1, 2, 3, or 4 independently selected R² substituents;
each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{2A} is independently selected from halo, C₁₋₆ alkyl, C₁₋₆ haloalkyl, C₂₋₆ alkenyl, and C₂₋₆ alkynyl;
R⁴ is selected from H and C₁₋₆ alkyl; and
R⁵ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

In some embodiments:
W is C(R^{W})₂;
Y is C(R^{Y})₂;
each R^{W} is independently selected from H, oxo, and C₁₋₃ alkyl;
each R^{Y} is independently selected from H, oxo, and C₁₋₃ alkyl;
Ring A is C₃₋₇ cycloalkyl, wherein the C₃₋₇ cycloalkyl of Ring A is optionally substituted with 1, 2, 3, or 4 independently selected R¹ substituents;
each R¹ is independently selected from C₁₋₆ alkyl, C₁₋₆ haloalkyl, NR^{c1}C(O)R^{b1}, NR^{c1}C(O)OR^{a1}, and NR^{c1}C(O)NR^{c1}R^{d1}, wherein each C₁₋₆ alkyl of R¹ is optionally substituted with 1, 2, 3, or 4 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, NR^{c11}C(O)R^{b11}, and NR^{c11}C(O)OR^{a11};
each R^{a11}, R^{b11}, and R^{d11} is independently selected from H, C₁₋₆ alkyl, and C₃₋₇ cycloalkyl;
Cy² is bicyclic 8-10 membered heteroaryl, wherein the bicyclic 8-10 membered heteroaryl of Cy² is optionally substituted with 1, 2, 3, or 4 independently selected R² substituents;
each R² is independently selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, and C₂₋₆ alkynyl;
R⁴ is selected from H and C₁₋₆ alkyl; and
R⁵ is selected from H and C₁₋₆ alkyl.

In some embodiments:
W is CH₂;
Y is CH₂;
Ring A is C₃₋₇ cycloalkyl, wherein the C₃₋₇ cycloalkyl of Ring A is optionally substituted with 1, 2, 3, or 4 independently selected R¹ substituents;
each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents;
each R^{c1} and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, NR^{c11}C(O)R^{b11}, and NR^{c11}C(O)OR^{a11};
each R^{a11}, R^{b11}, and R^{c11} is independently selected from H, C₁₋₆ alkyl, and C₃₋₇ cycloalkyl;
Cy² is bicyclic 8-10 membered heteroaryl, wherein the bicyclic 8-10 membered heteroaryl of Cy² is optionally substituted with 1, 2, 3, or 4 independently selected R² substituents;
each R² is independently selected from C₁₋₆ alkyl, C₁₋₆ haloalkyl, and 5-10 membered heteroaryl, wherein each C₁₋₆ alkyl and 5-10 membered heteroaryl of R² is optionally substituted with 1, 2, 3, or 4 independently selected R^{2A} substituents;
each R^{2A} is independently selected from C₁₋₆ alkyl and C₁₋₆ haloalkyl;
R⁴ is C₁₋₆ alkyl; and
R⁵ is H.

In some embodiments, the compound of Formula I is a compound of Formula II:

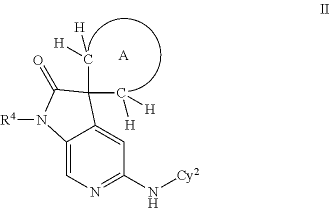

or a pharmaceutically acceptable salt thereof.

In some embodiments, the compound of Formula I is a compound of Formula III:

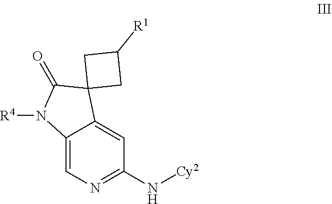

or a pharmaceutically acceptable salt thereof.

In some embodiments, the compound of Formula I is a compound of Formula IIIa:

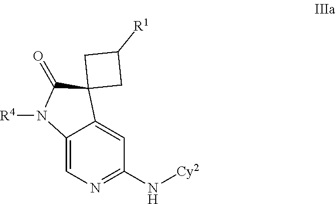

or a pharmaceutically acceptable salt thereof.

In some embodiments, the compound of Formula I is a compound of Formula IV:

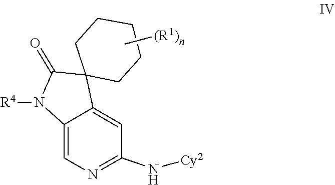

or a pharmaceutically acceptable salt thereof, wherein n is 0, 1, 2, 3, 4, 5, or 6.

In some embodiments, the compound of Formula I is a compound of Formula IVa:

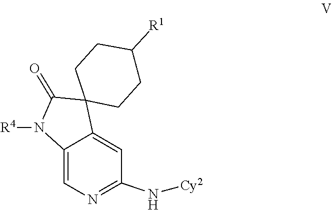

or a pharmaceutically acceptable salt thereof.

In some embodiments, the compound of Formula I is a compound of Formula Va:

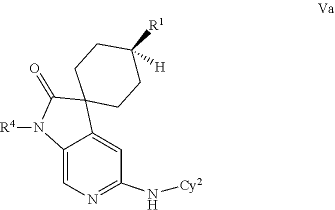

or a pharmaceutically acceptable salt thereof.

In some embodiments, the compound of Formula I is a compound of Formula Vb:

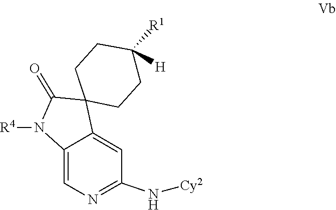

or a pharmaceutically acceptable salt thereof.

In some embodiments, the compound provided herein is selected from:
1-ethyl-3-((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea;
1-ethyl-3-((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea;
methyl (((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)carbamate;
N-(((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)cyclopropanecarboxamide;
(1r,3r)-3-(hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one;
methyl (((1r,3r)-1′-(methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)carbamate; and
N-(((1r,3r)-1′-(methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)cyclopropanecarboxamide;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “Cₒ₋ₚ alkoxy-Cₙ₋ₘ alkoxy-”, employed alone or in combination with other terms, refers to a group of formula (Cₒ₋ₚ alkyl)-O—(Cₙ₋ₘ alkyl)-O—, wherein the Cₒ₋ₚ alkyl has o to m carbons and the Cₙ₋ₘ alkyl has n to m carbons. Example Cₒ₋ₚ alkoxy-Cₙ₋ₘ alkoxy- groups include, but are not limited to, methoxymethoxy (i.e., CH₃OCH₂O—), methoxyethoxy (i.e., CH₃OCH₂CH₂O—), ethoxymethxoy (i.e., CH₃CH₂OCH₂O—), ethoxyethoxy (i.e., CH₃CH₂OCH₂CH₂O—), methoxypropoxy (i.e., CH₃OCH₂CH₂CH₂O—), and the like.

As used herein, the term “amino” refers to a group of formula-NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, C₁, or Br. In some embodiments, a halo is F or C₁. In some embodiments, a halo is F. In some embodiments, a halo is C₁.

As used herein, “Cₙ₋ₘ haloalkoxy” refers to a group of formula-O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “carbonyl”, employed alone or in combination with other terms, refers to a —C(O)— group.

As used herein, the term “Cₙ₋ₘ alkylcarbonyl” refers to a group of formula-C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula-S(O) 2-alkyl, wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)amino” refers to a group of formula-N (alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., —O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the(S)-configuration. The Formulas (e.g., Formula I, Formula IIa, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I can be prepared, for example, as shown in Scheme 1. An appropriately substituted starting material 1-1 wherein X¹ is a halogen or pseudohalogen (e.g., Cl, Br, I or OTf) can be treated with a base (e.g., LHMDS) and reacted with an appropriately substituted alkylating agent 1-2, wherein X² is a leaving group (e.g., Cl, Br, I, OMs, OTs), at a suitable temperature (e.g., from about −78° C. to 70° C.), to provide intermediate 1-3. Reaction of intermediate 1-3 with an alkylating agent 1-4, wherein X³ is a suitable leaving group (e.g., Cl, Br, I, OMs, OTs) in the presence of a base (e.g., Cs₂CO₃) affords intermediate 1-5. Cross-coupling of intermediate 1-5 with an amine 1-6, under conditions for Buchwald-Hartwig amination (e.g., in the presence of a catalyst such as tris(dibenzylideneacetone) dipalladium (0), a ligand such as 9,9-dimethyl-4,5-bis(diphenylphosphino) xanthene and a base such as Cs₂CO₃, at elevated temperature such as 80° C.) furnishes compounds of Formula I. Where suitable, an appropriate functional group on ring A can be subjected to further transformations at any stage in the synthesis deemed compatible with the intermediates and subsequent steps to be performed.

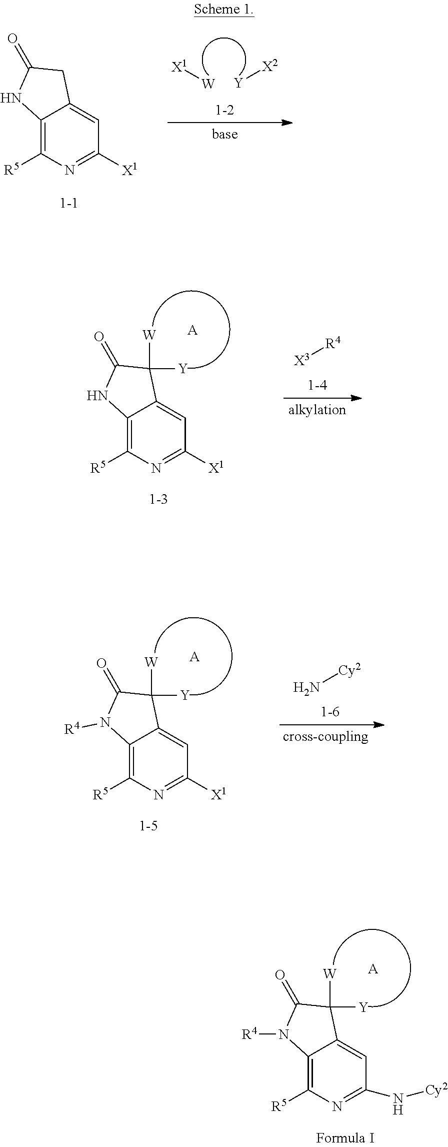

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure are useful in treating or preventing proliferative disorders such as cancers. In particular tumors with activating mutants of receptor tyrosine kinases or upregulation of receptor tyrosine kinases may be particularly sensitive to the inhibitors.

In certain embodiments, the disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11:49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥10% of cells in |
| multilineage dysplasia | 10⁹/L monocytes | ≥2 hematopoietic lineages, ±15% |
| | | ring sideroblasts, <5% |
| | | blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ± Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del (5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The compounds provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3^{rd} ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS—R, IGF-1R, IR—R, PDGFØR, PDGFBR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., parsaclisib and INCB50797), PI3K-gamma inhibitors such as PI3K-gamma selective inhibitors, MEK inhibitors, CSFIR inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibitors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US20170107216, US20170145025, U.S. US20170174671, US20170174679, US20170320875, US20170342060, US20170362253, and US20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab or utomilumab).

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil[bis (POM)-PMEA]; lobucavir (BMS-180194); BCH-10652; emitricitabine[(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms to allow the compound to be deuterated (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for-CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein (e.g., the compound of any of Formulas I-Vb), or a pharmaceutically acceptable salt thereof, comprises at least one deuterium atom.

In some embodiments, the compound provided herein (e.g., the compound of any of Formulas I-Vb), or a pharmaceutically acceptable salt thereof, comprises two or more deuterium atoms.

In some embodiments, the compound provided herein (e.g., the compound of any of Formulas I-Vb), or a pharmaceutically acceptable salt thereof, comprises three or more deuterium atoms.

In some embodiments, for a compound provided herein (e.g., the compound of any of Formulas I-Vb), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).

### Example 1. 1-Ethyl-3-((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea, trifluoroacetate salt (Single Diastereomer Prepared)

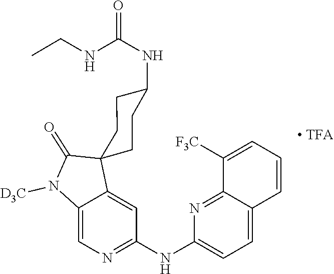 [SMILES: [2H]C([2H])([2H])N1C(=O)C2(CCC(NC(=O)NCC)CC2)c2cc(Nc3ccc4cccc(C(F)(F)F)c4n3)ncc21]

### Step 1. 5-Chlorodispiro[pyrrolo[2,3-c]pyridine-3,1′-cyclohexane-4′,2″-[1,3]dioxolan]-2(1H)-one

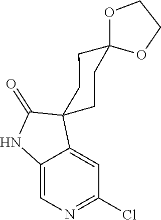 [SMILES: O=C1Nc2cnc(Cl)cc2C12CCC1(CC2)OCCO1]

To a suspension of 5-chloro-1,3-dihydro-2H-pyrrolo[2,3-c]pyridin-2-one (PharmaBlock #PB02480, 0.52 g, 3.1 mmol) in THF (9.5 mL) at −78° C. was added LHMDS (1.0 M in THF, 6.8 mL, 6.8 mmol) slowly dropwise. The solution was warmed to −50° C. for 30 min, then was re-cooled to −78° C. 2,2-Bis(2-bromoethyl)-1,3-dioxolane (1Click Chemistry #3C_{74174, 0.89} g, 3.1 mmol) was added, and the reaction was removed from the cooling bath and allowed to warm to room temperature and stir for 30 min. The reaction was then heated to 66° C. for 19 hrs. Upon cooling to room temperature, saturated NH₄Cl solution was added and the mixture was extracted with DCM (3×). The combined organic extracts were dried over Na₂SO₄, filtered and concentrated in vacuo. The product was purified via flash column chromatography, eluting with a gradient of 0-60% EtOAc in hexanes, to afford the title compound (0.20 g, 22%). LCMS for C₁₄H₁₆ClN₂O₃ (M+H)⁺: calculated monoisotopic m/z=295.1; found 295.1.

### Step 2. 5-Chloro-1-(methyl-d₃)dispiro[pyrrolo[2,3-c]pyridine-3,1′-cyclohexane-4′,2″-[1,3]dioxolan]-2(1H)-one

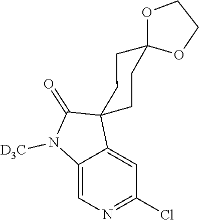 [SMILES: [2H]C([2H])([2H])N1C(=O)C2(CCC3(CC2)OCCO3)c2cc(Cl)ncc21]

A mixture of 5-chlorodispiro[pyrrolo[2,3-c]pyridine-3,1′-cyclohexane-4′,2″-[1,3]dioxolan]-2(1H)-one (from Step 1, 0.20 g, 0.68 mmol), Cs₂CO₃ (0.44 g, 1.4 mmol) and iodomethane-d₃ (0.051 mL, 0.81 mmol) in DMF (3.4 mL) was heated to 50° C. for 1 h. Upon cooling to room temperature, the reaction mixture was diluted with water and brine, and was extracted with EtOAc. The organic layer was washed with brine (2×), dried over Na₂SO₄, filtered and concentrated in vacuo. The product was purified via flash column chromatography, eluting with a gradient of 0-60% EtOAc in hexanes, to afford the title compound (0.18 g, 85%). LCMS for C₁₅H₁₅D₃ClN₂O₃ (M+H)⁺: calculated monoisotopic m/z=312.1; found 312.1.

### Step 3. 1-(Methyl-d₃)-5-((8-(trifluoromethyl)quinolin-2-yl)amino)dispiro[pyrrolo[2,3-c]pyridine-3,1′-cyclohexane-4′,2″-[1,3]dioxolan]-2(1H)-one

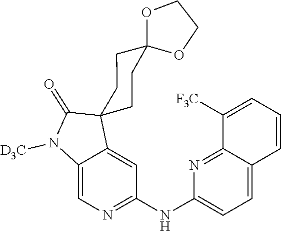 [SMILES: [2H]C([2H])([2H])N1C(=O)C2(CCC3(CC2)OCCO3)c2cc(Nc3ccc4cccc(C(F)(F)F)c4n3)ncc21]

A mixture of 5-chloro-1-(methyl-d₃)dispiro[pyrrolo[2,3-c]pyridine-3,1′-cyclohexane-4′,2″-[1,3]dioxolan]-2(1H)-one (from Step 2, 0.18 g, 0.58 mmol), 8-(trifluoromethyl)quinolin-2-amine (Aurum Pharmatech #GN26710, 0.37 g, 1.7 mmol), tris(dibenzylideneacetone) dipalladium (0) (0.079 g, 0.087 mmol), Cs₂CO₃ (0.75 g, 2.3 mmol) and 9,9-dimethyl-4,5-bis(diphenylphosphino) xanthene (0.10 g, 0.17 mmol) in dioxane (5.3 mL) was degassed by sparging with N₂ for 3 min. The reaction mixture was heated to 80° C. for 6 hrs. Upon cooling to room temperature, the reaction mixture was diluted with DCM and filtered through Celite®, rinsing with DCM. The filtrate was washed with water and the aqueous layer was further extracted with DCM (2×). The combined organic extracts were washed with brine, dried over Na₂SO₄, filtered and concentrated in vacuo. The product was purified via flash column chromatography, eluting with 100% EtOAc to afford the title compound (0.22 g, 78%). LCMS for C₂₅H₂₁D₃F₃N₄O₃ (M+H)⁺: calculated m/z=488.2; found 488.2.

### Step 4. 1′-(Methyl-d₃)-5′-((8-(trifluoromethyl)quinolin-2-yl)amino)spiro[cyclohexane-1,3′-pyrrolo[2,3-c]pyridine]-2′,4(1′H)-dione

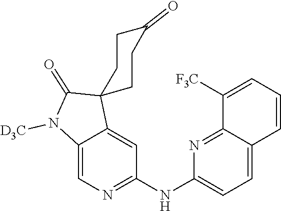 [SMILES: [2H]C([2H])([2H])N1C(=O)C2(CCC(=O)CC2)c2cc(Nc3ccc4cccc(C(F)(F)F)c4n3)ncc21]

1-(Methyl-d₃)-5-((8-(trifluoromethyl)quinolin-2-yl)amino)dispiro[pyrrolo[2,3-c]pyridine-3,1′-cyclohexane-4′,2″-[1,3]dioxolan]-2(1H)-one (from Step 3, 0.22 g, 0.45 mmol) in acetone (2.3 mL) and water (2.3 mL) was treated with c.HCl (0.38 mL, 4.4 mmol) and stirred for 4 hrs. Additional c.HCl (0.38 mL, 4.4 mmol) was added, and the reaction mixture was stirred for an additional 1 h. The reaction mixture was made basic by the cautious addition of saturated NaHCO₃ solution, and the aqueous mixture was extracted with DCM (3×). The combined organic extracts were dried over Na₂SO₄, filtered, and washed with 5% MeOH in DCM (20 mL). The filtrate was concentrated in vacuo to afford the title compound, which was used without further purification (0.19 g, 95%). LCMS for C₂₃H₁₇D₃F₃N₄O₂ (M+H)⁺: calculated m/z=444.2; found 444.3.

### Step 5. 4-Amino-1′-(methyl-d₃)-5′-((8-(trifluoromethyl)quinolin-2-yl)amino)spiro[cyclohexane-1,3′-pyrrolo[2, 3-c]pyridin]-2′(1′H)-one (Mixture of Diastereomers Prepared)

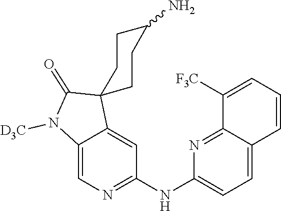 [SMILES: [2H]C([2H])([2H])N1C(=O)C2(CCC(N)CC2)c2cc(Nc3ccc4cccc(C(F)(F)F)c4n3)ncc21]

A suspension of 1′-(methyl-d₃)-5′-((8-(trifluoromethyl)quinolin-2-yl)amino)spiro[cyclohexane-1,3′-pyrrolo[2,3-c]pyridine]-2′,4 (1′H)-dione (from Step 4, 0.14 g, 0.28 mmol) in MeOH (4.7 mL) was stirred with ammonium acetate (0.29 g, 3.8 mmol) overnight. Sodium cyanoborohydride (0.062 g, 0.99 mmol) was then added and the mixture was stirred for 4 hrs. HCl solution (1.0 N, 2.0 mL, 2.0 mmol) was added, and the mixture was stirred for 20 min. MeOH was evaporated in vacuo. The residue was partitioned between 1.0 N NaOH and EtOAc. The aqueous portion was extracted with two further portions of EtOAc. The combined organic extracts were dried over Na₂SO₄, filtered and concentrated in vacuo to afford the title compound which was used without further purification (theoretical yield assumed). LCMS for C₂₃H₂₀D₃F₃N₅O (M+H)⁺: calculated m/z=445.2; found 445.2.

### Step 6. tert-Butyl ((1s, 4s)-1′-(methyl-d₃)-2′-oxo-5′-((8-(trifluoromethyl)quinolin-2-yl)amino)-1′, 2′-dihydrospiro[cyclohexane-1,3′-pyrrolo[2,3-c]pyridin]-4-yl)carbamate and tert-Butyl ((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)carbamate (Single Diastereomers Isolated)

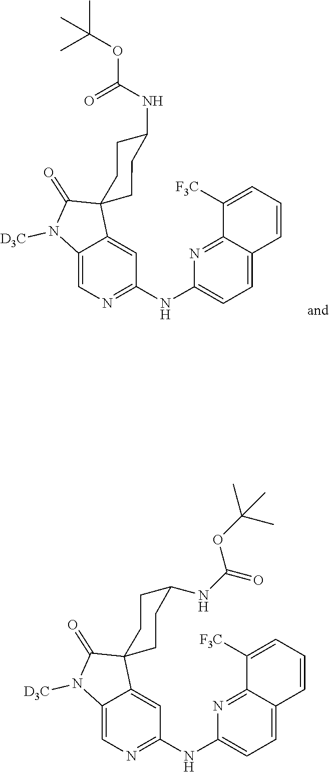 [SMILES: [2H]C([2H])([2H])N1C(=O)C2(CCC(NC(=O)OC(C)(C)C)CC2)c2cc(Nc3ccc4cccc(C(F)(F)F)c4n3)ncc21 ; [2H]C([2H])([2H])N1C(=O)C2(CCC(NC(=O)OC(C)(C)C)CC2)c2cc(Nc3ccc4cccc(C(F)(F)F)c4n3)ncc21]

To a solution of 4-amino-1′-(methyl-d₃)-5′-((8-(trifluoromethyl)quinolin-2-yl)amino)spiro[cyclohexane-1,3′-pyrrolo[2,3-c]pyridin]-2′(1′H)-one (mixture of diastereomers from Step 5, 0.12 g, 0.28 mmol) in THF (2.7 mL) was added a solution of NaHCO₃ (0.12 g, 1.4 mmol) in water (1.3 mL), followed by di-tert-butyl dicarbonate (0.12 g, 0.56 mmol). The mixture was stirred at room temperature overnight. The reaction mixture was diluted with water and extracted with EtOAc. The organic extract was washed with brine, dried over Na₂SO₄, filtered and concentrated in vacuo. The product was purified via flash column chromatography, eluting with a gradient of 0-80% EtOAc in hexanes, to afford the title compound (0.082 g, 66%). LCMS for C₂₈H₂₈D₃F₃N₅O₃ (M+H)⁺: calculated m/z=545.3; found 545.3. The diastereomers were separated via HPLC (Chiral Tech IG column, 5 μm, 21×250 mm, loading 15 mg in 3.6 mL EtOH per injection, eluting with 70% EtOH in hexanes at 20 mL/min for 18 min). The relative stereochemistry of the product obtained as Peak 1 was assigned by NMR of the product of Step 8. The relative stereochemistry of the product obtained as Peak 2 was assigned by NMR of the product of Example 2.

Peak 1 (first to elute, r.t.=6.2 min): tert-butyl ((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)carbamate (0.030 g, 20%).

Peak 2 (second to elute, r.t.=11.2 min): tert-butyl ((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)carbamate (0.033 g, 22%).

### Step 7. (1s,4s)-4-Amino-1′-(methyl-d₃)-5′-((8-(trifluoromethyl)quinolin-2-yl)amino)spiro[cyclohexane-1,3′-pyrrolo[2,3-c]pyridin]-2′(1′H)-one (Single Diastereomer Prepared)

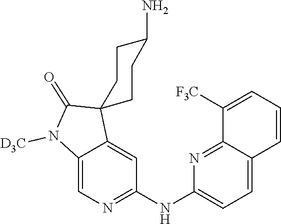 [SMILES: [2H]C([2H])([2H])N1C(=O)C2(CCC(N)CC2)c2cc(Nc3ccc4cccc(C(F)(F)F)c4n3)ncc21]

To a solution of tert-butyl ((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)carbamate (Peak 1 from Step 6, 0.030 g, 0.055 mmol) in DCM (0.55 mL) was added TFA (85 μL, 1.1 mmol) and the reaction mixture was stirred for 30 min. Volatiles were removed in vacuo. The residue was dissolved in DCM, and was made basic by the addition of saturated NaHCO₃. The layers were separated and the aqueous was extracted with DCM (2×). The combined organic extracts were dried over Na₂SO₄, filtered and concentrated in vacuo to afford the title compound which was used without further purification (0.023 g, 95%). LCMS for C₂₃H₂₀D₃F₃NO (M+H)⁺: calculated m/z=445.2; found 445.3.

### Step 8. 1-Ethyl-3-((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea, trifluoroacetate salt (Single Diastereomer Prepared)

To a solution of (1s,4s)-4-amino-1′-(methyl-d₃)-5′-((8-(trifluoromethyl)quinolin-2-yl)amino)spiro[cyclohexane-1,3′-pyrrolo[2,3-c]pyridin]-2′(1′H)-one (from Step 7, 8.0 mg, 0.018 mmol) and DIPEA (9.4 μL, 0.054 mmol) in DCM (0.18 mL) was added ethyl isocyanate (1.6 μL, 0.020 mmol). The reaction was stirred for 30 min at room temperature, then was concentrated in vacuo. The residue was dissolved in MeOH/MeCN, filtered and purified via preparative HPLC-MS (pH 2) to afford the title compound (6.7 mg, 53%). LCMS for C₂₆H₂₅D₃F₃N₆O₂ (M+H)⁺: calculated m/z=516.2; found 516.2. 1H NMR (500 MHZ, DMSO-d₆) δ 11.20 (s, 1H), 8.44 (d, J=9.0 Hz, 1H), 8.22-8.17 (m, 2H), 8.12 (d, J=7.5 Hz, 1H), 8.08 (s, 1H), 7.66 (d, J=9.0 Hz, 1H), 7.58 (t, J=7.7 Hz, 1H), 5.80 (m, 2H), 3.69-3.53 (m, 1H), 3.05 (q, J=7.2 Hz, 2H), 2.15-2.05 (m, 2H), 1.86-1.71 (m, 4H), 1.70-1.60 (m, 2H), 1.02 (t, J=7.1 Hz, 3H).

### Example 2. 1-Ethyl-3-((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea, trifluoroacetate salt (Single Diastereomer Prepared)

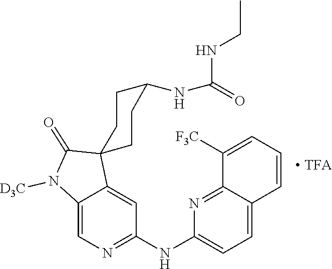 [SMILES: [2H]C([2H])([2H])N1C(=O)C2(CCC(NC(=O)NCC)CC2)c2cc(Nc3ccc4cccc(C(F)(F)F)c4n3)ncc21]

The procedure of Example 1, Steps 7 through 8 were followed, using tert-butyl ((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)carbamate (Peak 2 from Step 6 of Example 1, 0.033 g, 0.061 mmol) as starting material, to afford the title compound (5.2 mg, 12%). LCMS for C₂₆H₂₅D₃F₃N₆O₂ (M+H)⁺: calculated m/z=516.2; found 516.2. 1H NMR (500 MHZ, DMSO-d₆) δ 10.63 (s, 1H), 8.79 (s, 1H), 8.28 (d, J=9.0 Hz, 1H), 8.11 (d, J=8.0 Hz, 1H), 8.09-8.04 (m, 2H), 7.50 (t, J=7.7 Hz, 1H), 7.42 (d, J=9.0 Hz, 1H), 5.92 (s, 1H), 5.63 (s, 1H), 3.69-3.54 (m, 1H), 3.04 (q, J=7.2 Hz, 2H), 2.05-1.94 (m, 2H), 1.94-1.84 (m, 2H), 1.84-1.72 (m, 4H), 1.01 (t, J=7.1 Hz, 3H).

### Example 3. Methyl (((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)carbamate, trifluoroacetate salt (Single Diastereomer Prepared)

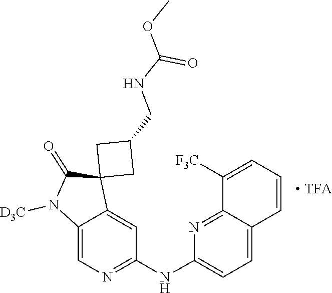 [SMILES: [2H]C([2H])([2H])N1c2cnc(Nc3ccc4cccc(C(F)(F)F)c4n3)cc2[C@]2(C[C@@H](CNC(=O)OC)C2)C1=O]

### Step 1. (3-Bromo-2-(bromomethyl)propoxy)(tert-butyl)diphenylsilane

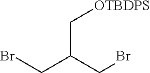

A solution of 3-bromo-2-(bromomethyl) propan-1-ol (prepared as in Carbohydrate Research (1987), 161 (2), pp. 225-233, 8.73 g, 37.6 mmol) and imidazole (2.69 g, 39.5 mmol) in DCM at 0° C. was treated with tert-butylchlorodiphenylsilane (10.3 mL, 39.5 mmol) and DMAP (0.460 g, 3.76 mmol). The reaction was allowed to warm to room temperature and stir for 48 h. The reaction mixture was diluted with diethyl ether and washed with water. The aqueous layer was extracted with an additional portion of diethyl ether. The combined organic extracts were washed with water, followed by brine, dried over Na₂SO₄, decanted and concentrated in vacuo. The product was purified via flash column chromatography, eluting with a gradient of 0-5% EtOAc in hexanes, to afford the title compound (14.1 g, 80.0%). LCMS for C₂₀H₂₇Br₂OSi (M+H)⁺: calculated monoisotopic m/z=469.0; found 469.1.

### Step 2. (1r,3r)-3-(((tert-Butyldiphenylsilyl)oxy)methyl)-5′-chlorospiro[cyclobutane-1,3′-pyrrolo[2,3-c]pyridin]-2′(1′H)-one (Single Diastereomer Prepared)

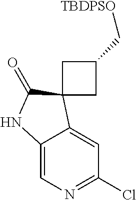

5-Chloro-1,3-dihydro-2H-pyrrolo[2,3-c]pyridin-2-one (PharmaBlock #PB02480, 1.6 g, 9.6 mmol) was azeotroped with toluene and then, as a suspension in THF (16 mL) at −78° C., was treated with LHMDS (1.0 M in THF, 22 mL, 22 mmol) slowly dropwise. The solution was warmed to −50° C. and stirred for 30 min, then was re-cooled to −78° C. A solution of (3-bromo-2-(bromomethyl)propoxy)(tert-butyl)diphenylsilane (from Step 1, also azeotroped with toluene, 3.0 g, 6.4 mmol) in THF (16 mL) was then added. The reaction mixture was first rapidly warmed by immersion in a water bath at room temperature, then was heated to reflux for 41 hrs. Upon cooling to room temperature, saturated NH₄Cl solution was added, and the mixture was extracted with EtOAc. The organic layer was washed with water, followed by brine, dried over Na₂SO₄, filtered and concentrated in vacuo. The product was purified via flash column chromatography, eluting with a gradient of 0-40% EtOAc in hexanes, to afford the title compound (0.51 g, 17%). LCMS for C₂₇H₃₀ClN₂O₂Si (M+H)⁺: calculated monoisotopic m/z=477.2; found 477.3. 1H NMR (500 MHZ, CDCl₃) δ 7.96 (s, 1H), 7.74-7.69 (m, 4H), 7.55-7.40 (m, 6H), 7.30 (s, 1H), 3.75 (d, J=4.5 Hz, 2H), 3.16-3.05 (m, 1H), 2.58-2.44 (m, 4H), 1.17 (s, 9H).

### Step 3. (1r,3r)-3-(((tert-Butyldiphenylsilyl)oxy)methyl)-5′-chloro-1′-(methyl-d₃)spiro[cyclobutane-1,3′-pyrrolo[2,3-c]pyridin]-2′(1′H)- one

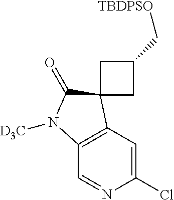

The procedure of Example 1, Step 2 was followed, using (1r,3r)-3-(((tert-butyldiphenylsilyl)oxy)methyl)-5′-chlorospiro[cyclobutane-1,3′-pyrrolo[2,3-c]pyridin]-2′(1′H)-one (from Step 2, 0.51 g, 1.1 mmol) as starting material, to afford the title compound (0.49 g, 93%). LCMS for C₂₈H₂₉D₃ClN₂O₂Si (M+H)⁺: calculated m/z=494.2; found 494.2.

### Step 4. (1r,3r)-3-(((tert-Butyldiphenylsilyl)oxy)methyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one

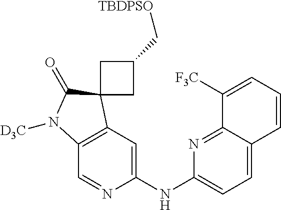

The procedure of Example 1, Step 3 was followed, using (1r,3r)-3-(((tert-butyldiphenylsilyl)oxy)methyl)-5′-chloro-1′-(methyl-d₃)spiro[cyclobutane-1,3′-pyrrolo[2,3-c]pyridin]-2′(1′H)-one (from Step 3, 0.14 g, 0.28 mmol) as starting material, to afford the title compound (0.16 g, 87%). LCMS for C₃₈H₃₅D₃F₃N₄O₂Si (M+H)⁺: calculated m/z=670.3; found 670.4.

### Step 5. (1r,3r)-3-(Hydroxymethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one

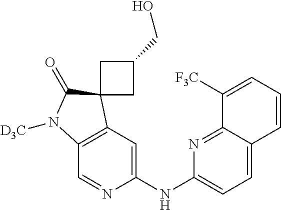 [SMILES: [2H]C([2H])([2H])N1c2cnc(Nc3ccc4cccc(C(F)(F)F)c4n3)cc2[C@]2(C[C@@H](CO)C2)C1=O]

A solution of (1r,3r)-3-(((tert-butyldiphenylsilyl)oxy)methyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one (from Step 4, 0.16 g, 0.24 mmol) in MeOH (1.2 mL) and THF (1.2 mL) was treated with NaOH solution (3.0 M, 0.41 mL, 1.2 mmol) at 50° C. for 40 min, then at 60° C. for 6 hrs. Upon cooling to room temperature, the reaction mixture was treated with saturated NH₄Cl solution (10.0 mL) and was extracted with DCM (3×). The combined organic extracts were dried over Na₂SO₄, filtered and concentrated in vacuo. The product was purified via flash column chromatography, eluting with a gradient of 0-100% EtOAc in hexanes, to afford the title compound (0.061 g, 58%). LCMS for C₂₂H₁₇D₃F₃N₄O₂ (M+H)⁺: calculated m/z=432.2; found 432.2.

### Step 6. ((1r, 3r)-1′-(Methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl methanesulfonate

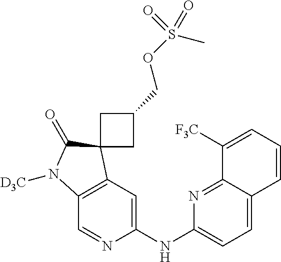 [SMILES: [2H]C([2H])([2H])N1c2cnc(Nc3ccc4cccc(C(F)(F)F)c4n3)cc2[C@]2(C[C@@H](COS(C)(=O)=O)C2)C1=O]

To a solution of (1r,3r)-3-(hydroxymethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one (from Step 5, 0.061 g, 0.14 mmol) and triethylamine (59 μL, 0.42 mmol) in DCM (1.0 mL) and DMF (1.0 mL) at 0° C. was added methanesulfonyl chloride (15 μL, 0.18 mmol). The reaction mixture was stirred for 40 min at 0° C., then was quenched by the addition of saturated NaHCO₃ solution. The mixture was diluted with brine and extracted with EtOAc. The organic layer was washed with brine (2×), dried over Na₂SO₄, filtered and concentrated in vacuo to afford the title compound which was used without further purification in Step 6 (0.072 g, 100%). LCMS for C₂₃H₁₉D₃F₃N₄O₄S (M+H)⁺: calculated m/z=510.1; found 510.2.

### Step 7. (1r,3r)-3-(Aminomethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one

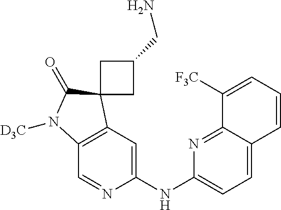 [SMILES: [2H]C([2H])([2H])N1c2cnc(Nc3ccc4cccc(C(F)(F)F)c4n3)cc2[C@]2(C[C@@H](CN)C2)C1=O]

A mixture of ((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl methanesulfonate (from Step 6, 0.072 g, 0.14 mmol) and NH₄OH (28% w/w, 0.55 mL, 8.0 mmol) in MeOH (1.4 mL) was heated at 80° C. for 16 hrs. Volatiles were removed in vacuo and the residue was dissolved in MeOH and filtered. The product was purified via preparative HPLC-MS (pH 2) and the eluent was evaporated. The residue was treated with saturated NaHCO₃ and extracted with DCM (3×). The combined organic extracts were dried over Na₂SO₄, filtered and concentrated in vacuo to afford the title compound (0.016 g, 26%). LCMS for C₂₂H₁₈D₃F₃N₅O (M+H)⁺: calculated m/z=431.2; found 431.2.

### Step 8. Methyl (((1r, 3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)carbamate, trifluoroacetate salt (Single Diastereomer Prepared)

A mixture of (1r,3r)-3-(aminomethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one (from Step 7, 4.0 mg, 9.3 μmol) and DIPEA (4.9 μL, 0.028 mmol) in DMF (0.10 mL) was added methyl chloroformate (1.0 mg, 10. μmol) as a solution in DCM (0.10 mL). After stirring for 1 h, the reaction mixture was diluted with MeOH and the product was purified via preparative HPLC-MS (pH 2) to afford the title compound (4.1 mg, 62%). LCMS for C₂₄H₂₀D₃F₃N₅O₃ (M+H)⁺: calculated m/z=489.2; found 489.2.

### Example 4. N-(((1r,3r)-1′-(Methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)cyclopropanecarboxamide, trifluoroacetate salt (Single Diastereomer Prepared)

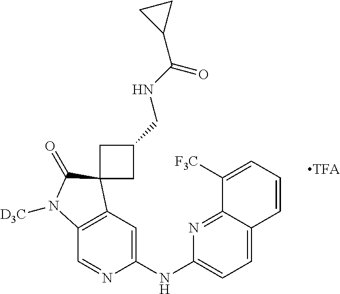 [SMILES: [2H]C([2H])([2H])N1c2cnc(Nc3ccc4cccc(C(F)(F)F)c4n3)cc2[C@]2(C[C@@H](CNC(=O)C3CC3)C2)C1=O]

The title compound was prepared by the method of Example 3, substituting cyclopropanecarbonyl chloride (1.0 mg, 10. μmol) in place of methyl chloroformate in Step 8 (4.2 mg, 62%). LCMS for C₂₆H₂₂D₃F₃N₅O₂ (M+H)⁺: calculated m/z=499.2; found 499.3.

### Example 5. (1r,3r)-3-(Hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one, trifluoroacetate salt (Single Diastereomer Prepared)

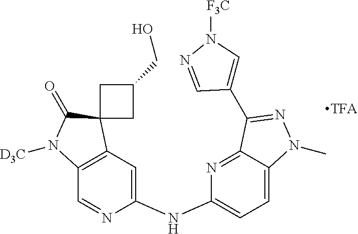 [SMILES: [2H]C([2H])([2H])N1c2cnc(Nc3ccc4c(n3)c(-c3cnn(C(F)(F)F)c3)nn4C)cc2[C@]2(C[C@@H](CO)C2)C1=O]

### Step 1. 5-Chloro-3-iodo-1-methyl-1H-pyrazolo[4,3-b]pyridine and 5-Chloro-3-iodo-2-methyl-2H-pyrazolo[4,3-b]pyridine (mixture of regioisomers prepared)

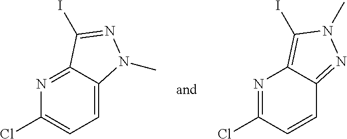

To a solution of 5-chloro-3-iodo-1H-pyrazolo[4,3-b]pyridine (PharmaBlock #PBZ3194, 1.3 g, 4.5 mmol) in DMF (16 mL) was added Cs₂CO₃ (2.9 g, 9.0 mmol), followed by iodomethane (0.42 mL, 6.7 mmol). The resulting mixture was stirred at 23° C. for 16 hrs. The reaction mixture was partitioned between EtOAc and water. The organic layer was washed with brine (3×), dried over Na₂SO₄, filtered and concentrated in vacuo to afford the title compounds as a mixture of regioisomers which was used without further purification (1.3 g, 49%). LCMS for C₇H₆ClIN₃ (M+H)⁺: calculated monoisotopic m/z=293.9; found 294.0.

### Step 2. 5-Chloro-1-methyl-3-(1-(trifluoromethyl)-1H-pyrazol-4-yl)-1H-pyrazolo[4,3-b]pyridine (single regioisomer isolated)

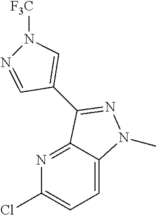 [SMILES: Cn1nc(-c2cnn(C(F)(F)F)c2)c2nc(Cl)ccc21]

A degassed mixture of 5-chloro-3-iodo-1-methyl-1H-pyrazolo[4,3-b]pyridine and 5-chloro-3-iodo-2-methyl-2H-pyrazolo[4,3-b]pyridine (mixture of regioisomers from Step 1, 1.3 g, 4.4 mmol), 4-(4,4,5,5-tetramethyl-1,3,2-dioxaborolan-2-yl)-1-(trifluoromethyl)-1H-pyrazole (1Click Chemistry #2C_{42502, 1.4} mL, 6.6 mmol), K₃PO₄ (2.8 g, 13 mmol) and dichlorobis(triphenylphosphine) palladium (II) (0.31 g, 0.44 mmol) in dioxane (35 mL) and water (9.3 mL) was heated at 60° C. for 1 h. Upon cooling to room temperature, the reaction mixture was diluted with EtOAc and washed with water. The organic layer was dried over Na₂SO₄, filtered and concentrated in vacuo. The crude mixture was purified via flash column chromatography, eluting with a gradient of 0-50% EtOAc in hexanes, to afford the title compound (0.68 g, 51%). LCMS for C₁₁H₈ClF₃N₅ (M+H)⁺: calculated monoisotopic m/z=302.0; found 302.1.

### Step 3. N-(1-Methyl-3-(1-(trifluoromethyl)-1H-pyrazol-4-yl)-1H-pyrazolo[4,3-b]pyridin-5-yl)-1,1-diphenylmethanimine

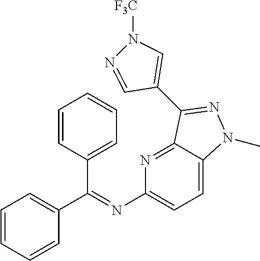 [SMILES: Cn1nc(-c2cnn(C(F)(F)F)c2)c2nc(N=C(c3ccccc3)c3ccccc3)ccc21]

A mixture of 5-chloro-1-methyl-3-(1-(trifluoromethyl)-1H-pyrazol-4-yl)-1H-pyrazolo[4,3-b]pyridine (from Step 2, 0.68 g, 2.3 mmol), Cs₂CO₃ (1.8 g, 5.6 mmol), Pd(OAc) 2 (0.051 g, 0.23 mmol), xantphos (0.20 g, 0.34 mmol) and benzophenone imine (0.57 mL, 3.4 mmol) in dioxane (15 mL) was degassed by sparging with nitrogen. The reaction mixture was heated to 100° C. for 1 h. Upon cooling to room temperature, the reaction mixture was diluted with EtOAc and was filtered. The filter was rinsed with EtOAc and the filtrate was concentrated in vacuo. The product was purified via flash column chromatography, eluting with a gradient of 0-40% EtOAc in hexanes, to afford the title compound (0.62 g, 62%). LCMS for C₂₄H₁₈F₃N₆ (M+H)⁺: calculated m/z=447.2; found 447.2.

### Step 4. 1-Methyl-3-(1-(trifluoromethyl)-1H-pyrazol-4-yl)-1H-pyrazolo[4,3-b]pyridin-5-amine

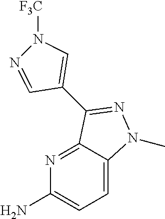 [SMILES: Cn1nc(-c2cnn(C(F)(F)F)c2)c2nc(N)ccc21]

To a solution of N-(1-methyl-3-(1-(trifluoromethyl)-1H-pyrazol-4-yl)-1H-pyrazolo[4,3-b]pyridin-5-yl)-1,1-diphenylmethanimine (from Step 3, 0.62 g, 1.4 mmol) in THF (8.7 mL) was added aq. HCl (1.0 N, 6.9 mL, 6.9 mmol). The reaction mixture was stirred at room temperature for 1 h. Saturated NaHCO₃ solution was added, and the product was extracted with DCM (2×). The combined organic extracts were dried over Na₂SO₄, filtered and concentrated in vacuo. The product was purified via flash column chromatography, eluting with a gradient of 0-5% MeOH in DCM to afford the title compound (0.36 g, 91%). LCMS for C₁₁H₁₀F₃N₆ (M+H)⁺: calculated m/z=283.1; found 283.2.

### Step 5. (1r,3r)-3-(((tert-Butyldiphenylsilyl)oxy)methyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one

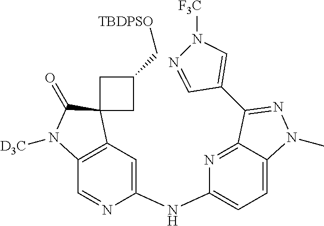

A mixture of (1r,3r)-3-(((tert-butyldiphenylsilyl)oxy)methyl)-5′-chloro-1′-(methyl-d₃)spiro[cyclobutane-1,3′-pyrrolo[2,3-c]pyridin]-2′(1′H)-one (from Example 3, Step 3, 0.15 g, 0.30 mmol), tris(dibenzylideneacetone) dipalladium (0) (0.041 g, 0.045 mmol), 1-methyl-3-(1-(trifluoromethyl)-1H-pyrazol-4-yl)-1H-pyrazolo[4,3-b]pyridin-5-amine (from Step 4 of Example 5, 0.10 g, 0.36 mmol), Cs₂CO₃ (0.39 g, 1.2 mmol) and 9,9-dimethyl-4,5-bis(diphenylphosphino) xanthene (0.052 g, 0.089 mmol) in dioxane (2.7 mL) was degassed by sparging with N₂. The reaction was heated to 80° C. for 7 hrs. Upon cooling to room temperature, the reaction mixture was diluted with EtOAc and filtered through Celite®. The filtrate was washed with water, followed by brine, was dried over Na₂SO₄, filtered and concentrated in vacuo. The product was purified via flash column chromatography, eluting with 100% EtOAc, to afford the title compound (0.22 g, 100%). LCMS for C₃₉H₃₇D₃F₃N₈O₂Si (M+H)⁺: calculated m/z=740.3; found 740.4.

### Step 6. (1r,3r)-3-(Hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one, trifluoroacetate salt (Single Diastereomer Prepared)

To a solution of (1r,3r)-3-(((tert-butyldiphenylsilyl)oxy)methyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one (from Step 5, 0.22 g, 0.30 mmol) in MeOH (1.5 mL) and THF (1.5 mL) was added aq. NaOH (3.0 N, 0.50 mL, 1.50 mmol). The reaction mixture was heated to reflux for 2 hrs. Upon cooling to room temperature, saturated NH₄Cl was added and the mixture was extracted with 10% iPrOH in DCM (3×). The combined organic extracts were dried over Na₂SO₄, filtered and concentrated in vacuo. The product was purified via flash column chromatography, eluting with a gradient of 0-10% MeOH in DCM, to afford the title compound (0.10 g, 69%). Further purification of the alcohol via preparative HPLC-MS (pH 2) afforded the title compound as the trifluoroacetate salt. LCMS for C₂₃H₁₉D₃F₃N₈₀₂ (M+H)⁺: calculated m/z=502.2; found 502.3.

### Example 6. Methyl (((1r,3r)-1′-(methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)carbamate, trifluoroacetate salt (Single Diastereomer Prepared)

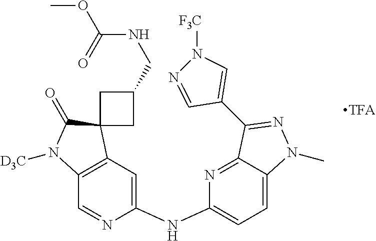 [SMILES: [2H]C([2H])([2H])N1c2cnc(Nc3ccc4c(n3)c(-c3cnn(C(F)(F)F)c3)nn4C)cc2[C@]2(C[C@@H](CNC(=O)OC)C2)C1=O]

The procedure of Example 3, Steps 6 through 8 were followed, using (1r,3r)-3-(hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one (from Example 5) as starting material in place of (1r,3r)-3-(hydroxymethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one in Step 6. LCMS for C₂₅H₂₂D₃F₃N₉O₃ (M+H)⁺: calculated m/z=559.2; found 559.3. ¹H NMR (400 MHZ, DMSO-d₆) δ 10.33 (s, 1H), 8.95 (s, 1H), 8.70 (s, 1H), 8.17 (d, J=9.1 Hz, 1H), 8.07 (s, 1H), 7.92 (s, 1H), 7.48 (d, J=9.1 Hz, 1H), 7.11 (s, 1H), 4.10 (s, 3H), 3.49 (s, 3H), 3.15-3.07 (m, 2H), 2.98-2.83 (m, 1H), 2.48-2.41 (m, 2H), 2.23-2.13 (m, 2H).

### Example 7. N-(((1r,3r)-1′-(Methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)cyclopropanecarboxamide, trifluoroacetate salt (Single Diastereomer Prepared)

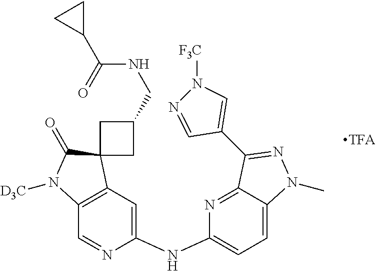 [SMILES: [2H]C([2H])([2H])N1c2cnc(Nc3ccc4c(n3)c(-c3cnn(C(F)(F)F)c3)nn4C)cc2[C@]2(C[C@@H](CNC(=O)C3CC3)C2)C1=O]

The procedure of Example 3, Steps 6 through 8 were followed, using (1r,3r)-3-(hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one (from Example 5) as starting material in place of (1r,3r)-3-(hydroxymethyl)-1′-(methyl-d₃)-5′-((8-(trifluoromethyl)quinolin-2-yl)amino)spiro[cyclobutane-1,3′-pyrrolo[2,3-c]pyridin]-2′(1′H)-one in Step 6, and substituting cyclopropanecarbonyl chloride for methyl chloroformate in Step 8. LCMS for C₂₇H₂₄D₃F₃N₉₀₂ (M+H)⁺: calculated m/z=569.2; found 569.3. ¹H NMR (400 MHZ, DMSO-d₆) δ 10.30 (s, 1H), 8.95 (s, 1H), 8.70 (s, 1H), 8.17 (d, J=9.2 Hz, 1H), 8.06 (s, 1H), 8.05-8.00 (m, 1H), 7.92 (s, 1H), 7.51 (d, J=9.2 Hz, 1H), 4.10 (s, 3H), 3.24-3.08 (m, 2H), 2.96-2.79 (m, 1H), 2.50-2.44 (m, 2H), 2.23-2.13 (m, 2H), 1.53-1.44 (m, 1H), 0.68-0.55 (m, 4H).

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM Fluorescent JAK2-JH1 Tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example B. JAK2 LanthaScreen JH2-WT Binding Assay

JAK2 JH2-WT binding assay utilizes pseudo-kinase domain (JH2, amino-acids 536-812 with 3 surface mutations W659A, W777A, F₇₉₄H) of human Wild Type JAK2 expressed as C-terminal His-Avi-tagged, biotinylated protein in a baculovirus expression system (BPS Bioscience, Catalog #79463). The assay was conducted in black 384-well polystyrene plates in a final reaction volume of 20 μL. JAK2 JH2-WT (0.145 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example C. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F₇₉₄H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example D. JAK2 HTRF Enzyme Activity Assay

JAK2 enzyme activity assays utilize catalytic domain (JH1, amino acids 808-1132) of human JAK2 expressed as N-terminal His-tagged protein in a baculovirus expression system (BPS Bioscience, Catalog #40450). The assays was conducted in black 384-well polystyrene plates in a final reaction volume of 20 μL. JAK2 (0.015 nM) was incubated with compounds (100 nL serially diluted in DMSO) in the presence of ATP (30 μM or 1 mM) and 500 nM Biotin-labeled EQEDEPEGDYFEWLE (SEQ ID NO.: 1) peptide (BioSource International, custom synthesis) in assay buffer (50 mM Tris, pH=7.5, 10 mM MgCl₂, 0.01% Brij-35, 0.1% BSA, 1 mM EGTA, 5% Glycerol and 5 mM DTT) for 60 minutes at 25° C. The reactions were stopped by the addition of 10 μL of detection buffer (50 mM Tris, pH 7.8, 0.5 mg/mL BSA, 150 mM NaCl), supplemented with EDTA, LANCE Eu-W1024 anti-phosphotyrosine (PY20), (PerkinElmer, Catalog #AD0067) and Streptavidin SureLight APC (PerkinElmer Catalog #CR130-100), for a final concentration of 15 mM, 1.5 nM and 75 nM, respectively. HTRF signals were read after 30 minutes incubation at room temperature on a PHERAstar FS plate reader (BMG LABTECH). Data was analyzed with IDBS XLfit and GraphPad Prism 5.0 software using a four parameter dose response curve to determine IC50 for each compound.

The compounds of the disclosure were tested in one or more of the assays described in Examples A-D, and the resulting data are shown in Table A.

TABLE A

| | JH2 BINDING | JH2 BINDING | JH1 | |
|---|---|---|---|---|
| Ex. | V617F | WT | BINDING | ENZ 30UMATP |
| No. | IC₅₀ (nM) | IC₅₀ (nM) | IC₅₀ (nM) | IC₅₀ (nM) |
| 1 | + | + | +++++ | +++++ |
| 2 | + | + | +++++ | +++++ |
| 3 | + | | | +++++ |
| 4 | + | | | +++++ |
| 5 | + | | | +++++ |
| 6 | + | | | +++++ |
| 7 | + | | | +++++ |

+ refers to IC₅₀ of ≤100 nM
++ refers to IC₅₀ of >100 nM to ≤500 nM
+++ refers to IC₅₀ of >500 nM to ≤1000 nM
++++ refers to IC₅₀ of >1000 nM to ≤2500 nM
+++++ refers to IC₅₀ of >2500 nM

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 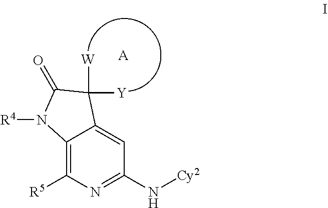
or a pharmaceutically acceptable salt thereof, wherein:
W is CR^{W} or C(R^{W})₂;
Y is CR^{Y} or C(R^{Y})₂;
each R^{W} is independently selected from H, oxo, C₁₋₆ alkyl, C₁₋₆ haloalkyl, C₂₋₆ alkenyl, and C₂₋₆ alkynyl;
each R^{Y} is independently selected from H, oxo, C₁₋₆ alkyl, C₁₋₆ haloalkyl, C₂₋₆ alkenyl, and C₂₋₆ alkynyl;
Ring A is selected from C₃₋₁₀ cycloalkyl and 4-10 membered heterocycloalkyl, wherein the C₃₋₁₀ cycloalkyl and 4-10 membered heterocycloalkyl of Ring A are each optionally substituted with 1, 2, 3, 4, 5, 6, 7, or 8 independently selected R¹ substituents;
each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a1}, SR^{a1}, NHOR^{a1}, C(O)R^{b1}, C(O)NR^{c1}R^{d1}, C(O)NR^{c1} (OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1}) R^{b1}, C(═NR^{e1}) NR^{c1}R^{d1}, NR^{c1}C(═NR^{e1}) NR^{c1}R^{d1}, NR^{c1}C(═NR^{e1}) R^{b1}, NR^{c1}S(O)R^{b1}, NR^{c1}S(O)NR^{c1}R^{d1}, NR^{c1}S(O)₂R^{b1}, NR^{c1}S(O)(═NR^{e1}) R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1}) R^{b1}, and OS(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{c1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11}) R^{b11}, C(═NR^{e11}) NR^{c11}R^{d11}, NR^{c11}C(═NR^{e11}) NR^{c11}R^{d11}, NR^{c11}C(═NR^{e11}) R^{b11} NR^{c11}S(O)R^{b11}, NR^{c11}S(O)NR^{c11}R^{d11}, NR^{c11}S(O)₂R^{b11}, NR^{c11}S(O)(═NR^{e11}) R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11}) R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁-6 alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, 4, 5, 6, 7, or 8 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R^{1B} substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1B} substituents;
each R^{b1}1 is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b11} are each optionally substituted with 1, 2, 3, 4, 5, 6, 7, or 8 independently selected R^{1B} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, SR^{a12}, NHOR^{a12}, C(O)R^{b12}, C(O)NR^{c12}R^{d12}, C(O)NR^{c12} (OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12}) R^{b12}, C(═NR^{e12}) NR^{c12}R^{d12}, NR^{c12}C(═NR^{e12}) NR^{c12}R^{d12}, NR^{c12}C(═NR^{e12}) R^{b12}, NR^{c12}S(O)R^{b12}, NR^{c12}S(O)NR^{c12}R^{d12}, NR^{c12}S(O)₂R^{b12}, NR^{c12}S(O)(═NR^{e12}) R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12}) R^{b12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl- and (4-7 membered heterocycloalkyl)-C₁₋₆ alkyl- of R^{1B} are each optionally substituted with 1, 2, 3, or 4 independently selected R^{1C} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a12}, R^{c12} and R^{d12} are each optionally substituted with 1, 2, 3, or 4 independently selected R^{1C} substituents;
or, any R^{c12} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{1C} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{1C} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a13}, SR^{a13}, NHOR^{a13}, C(O)R^{b13}, C(O)NR^{c13}R^{d13}, C(O)NR^{c13} (OR^{a13}), C(O)OR^{a13}, OC(O)R^{b13}, OC(O)NR^{c13}R^{d13}, NR^{c13}R^{d13}, NR^{c13}NR^{c13}R^{d13}, NR^{c13}C(O)R^{b13}, NR^{c13}C(O)OR^{a13}, NR^{c13}C(O)NR^{c13}R^{d13}, C(═NR^{e13}) R^{b13}, C(═NR^{e13}) NR^{c13}R^{d13}, NR^{c13}C(═NR^{e13}) NR^{c13}R^{d13}, NR^{c13}C(═NR^{e13}) R^{b13}, NR^{c13}S(O)R^{b13} NR^{c13}S(O)NR^{c13}R^{d13}, NR^{c13}S(O)₂R^{b13}, NR^{e13}S(O)(═NR^{e13}) R^{b13}, NR^{c13}S(O)₂NR^{c13}R^{d13}, S(O)R^{b13}, S(O)NR^{c13}R^{d13}, S(O)₂R^{b13}, S(O)₂NR^{c13}R^{d13}, OS(O)(═NR^{e13}) R^{b13}, and OS(O)₂R^{b13}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl- and (4-7 membered heterocycloalkyl)-C₁₋₆ alkyl- of R^{1C} are each optionally substituted with 1, 2, 3, or 4 independently selected R^{M} substituents;
each R^{a13}, R^{c13}, and R^{d13} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a13}, R^{c13} and R^{d13} are each optionally substituted with 1, 2, 3, or 4 independently selected R^{M} substituents;
or, any R^{c13} and R^{d13} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b13} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b13} are each optionally substituted with 1, 2, 3, or 4 independently selected R^{M} substituents;
each R^{e13} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
Cy² is selected from C₆₋₁₀ aryl, C₃₋₁₀ cycloalkyl, 5-10 membered heteroaryl, and 4-10 membered heterocycloalkyl, wherein the C₆₋₁₀ aryl, C₃₋₁₀ cycloalkyl, 5-10 membered heteroaryl, and 4-10 membered heterocycloalkyl of Cy² are each optionally substituted with 1, 2, 3, 4, 5, 6, 7, or 8 independently selected R² substituents;
each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{2C} (O)R^{b2}, NR^{2C} (O)OR^{a2}, NR^{c2}C(O)NR^{c2}R^{d2}, C(═NR^{e2})R^{b2}, C(═NR^{e2}) NR^{c2}R^{d2}, NR^{c2}C(═NR^{e2}) NR^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, and OS(O)₂R^{b2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{e2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{e2} and R^{d2} are each optionally substituted with 1, 2, 3, 4, 5, 6, 7, or 8 independently selected R^{2A} substituents;
or, any R^{e2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21} (OR^{a21}), C(O)OR^{a21}, OC(O)R^{b21}, OC(O)NR^{c21}R^{d21}, NR^{c21}R^{d21} NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21}) R^{b21}, C(═NR^{e21}) NR^{c21}R^{d21}, NR^{c21}C(═NR^{e21}) NR^{c21}R^{d21}, NR^{c21}C(═NR^{e21}) R^{b21}, NR^{c21}S(O)R^{b21}, NR^{c21}S(O)NR^{c21}R^{d21}, NR^{c21}S(O)₂R^{b21}, NR^{c21}S(O)(═NR^{e21}) R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{e21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22} (OR^{a22}), C(O)OR^{a22}, OC(O)R^{b22}, OC(O)NR^{e22}R^{d22}, NR^{c22}R^{d22}, NR^{c22}NR^{c22}R^{d22}, NR^{c22}C(O)R^{b22}, NR^{c22}C(O)OR ^{a22}, NR^{c22}C(O)NR^{c22}R^{d22}, C(═NR^{e22}) R^{b22}, C(═NR^{e22}) NR^{c22}R^{d22}, NR^{c22}C(═NR^{e22}) NR^{c22}R^{d22}, NR^{c22}C(═NR^{e22}) R^{b22}, NR^{c22}S(O)R^{b22}, NR^{c22}S(O)NR^{c22}R^{d22}, NR^{c22}S(O)₂R^{b22}, NR^{c22}S(O)(═NR^{e22}) R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22}) R^{b22}, and OS(O)₂R^{b22}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁-6 alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2B} are each optionally substituted with 1, 2, 3, 4, 5, 6, 7, or 8 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a22}, R^{c22} and R^{d22} are each optionally substituted with 1, 2, 3, 4, 5, 6, 7, or 8 independently selected R^{2C} substituents;
or, any R^{e22} and R^{d22} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2C} substituents;
each R^{b22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b22} are each optionally substituted with 1, 2, 3, 4, 5, 6, 7, or 8 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a23}, SR^{a23}, NHOR^{a23}, C(O)R^{b23}, C(O)NR^{c23}R^{d23}, C(O)NR^{c23} (OR^{a23}), C(O)OR^{a23}, OC(O)R^{b23}, OC(O)NR^{c23}R^{d23}, NR^{c23}R^{d23} NR^{c23} NR^{c23}R^{d23}, NR^{c23}C(O)R^{b23}, NR^{c23}C(O)OR^{a23}, NR^{c23}C(O)NR^{c23}R^{d23}, C(═NR^{e23}) R^{b23}, C(═NR^{e23}) NR^{c23}R^{d23}, NR^{c23}C(═NR^{e23}) NR^{c23}R^{d23}, NR^{c23} C(═NR^{e23}) R^{b23}, NR^{c23}S(O)R^{b23}, NR^{c23}S(O)NR^{c23}R^{d23}, NR^{c23} S(O)₂R^{b23}, NR^{c23}S(O)(═NR^{e23}) R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, OS(O)(═NR^{e23}) R^{b23}, and OS(O)₂R^{b23}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a23}, R^{e23}, and R^{d23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a23}, R^{c23} and R^{d23} are each optionally substituted with 1, 2, 3, or 4 independently selected R^{M} substituents;
or, any R^{c23} and R^{d23} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b23} are each optionally substituted with 1, 2, 3, or 4 independently selected R^{M} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a5}, NH₂, and NHC₁₋₆ alkyl, wherein the C₁₋₆ alkyl is optionally substituted with OH, CN, and NH₂;
R^{a5} is H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂; and
each R^{M} is independently selected from H, OH, halo, oxo, CN, C(O)OH, C(O)NH₂, C(O)NH(C₁₋₄ alkyl), C(O)N(C₁₋₆ alkyl) ₂, NH₂, NO₂, SF5, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-.

2. The compound of claim 1, or a pharmaceutically acceptable salt thereof, wherein W is C(R^{W})₂.

3. The compound of claim 1, or a pharmaceutically acceptable salt thereof, wherein W is CH₂.

4. The compound of claim 1, or a pharmaceutically acceptable salt thereof, wherein Y is C(R^{Y})₂.

5. The compound of claim 1, or a pharmaceutically acceptable salt thereof, wherein Y is CH₂.

6. The compound of claim 1, or a pharmaceutically acceptable salt thereof, wherein Ring A is C₃₋₁₀ cycloalkyl, wherein the C₃₋₁₀ cycloalkyl of Ring A is optionally substituted with 1, 2, 3, or 4 independently selected R¹ substituents.

7. The compound of claim 1, or a pharmaceutically acceptable salt thereof, wherein Ring A is C₃₋₇ cycloalkyl, wherein the C₃₋₇ cycloalkyl of Ring A is optionally substituted with 1, 2, 3, or 4 independently selected R¹ substituents.

8. The compound of claim 1, or a pharmaceutically acceptable salt thereof, wherein Ring A is selected from cyclobutyl and cyclohexyl, wherein the cyclobutyl and cyclohexyl of Ring A are each optionally substituted with 1, 2, 3, or 4 independently selected R¹ substituents.

9. The compound of claim 1, or a pharmaceutically acceptable salt thereof, wherein each R¹ is independently selected from C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, NR^{c1}C(O)R^{b1}, NR^{c1}C(O)OR^{a1}, and NR^{c1}C(O)NR^{c1}R^{d1}, wherein the C₁₋₆ alkyl, C₂₋₆ alkenyl, and C₂₋₆ alkynyl of R¹ are each optionally substituted with 1, 2, 3, or 4 independently selected R^{1A} substituents.

10. The compound of claim 1, or a pharmaceutically acceptable salt thereof, wherein each R¹ is independently selected from C₁₋₆ alkyl, C₁₋₆ haloalkyl, NR^{c1}C(O)R^{b1}, NR^{c1}C(O)OR^{a1}, and NR^{c1}C(O)NR^{c1}R^{d1}, wherein each C₁₋₆ alkyl of R¹ is optionally substituted with 1, 2, 3, or 4 independently selected R^{1A} substituents.

11. The compound of claim 1, or a pharmaceutically acceptable salt thereof, wherein each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents; and
each R^{c1} and R^{d1} is independently selected from H, C₁₋₆ alkyl, C₁₋₆ haloalkyl, C₂₋₆ alkenyl, and C₂₋₆ alkynyl.

12. The compound of claim 1, or a pharmaceutically acceptable salt thereof, wherein each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents; and
each R^{c1} and R^{d1} is independently selected from H and C₁₋₆ alkyl.

13. The compound of claim 1, or a pharmaceutically acceptable salt thereof, wherein each R¹ is independently selected methyl and —NHC(O)NHCH₂CH₂, wherein each methyl of R¹ is optionally substituted with 1, 2, 3, or 4 independently selected R^{1A} substituents.

14. The compound of claim 1, or a pharmaceutically acceptable salt thereof, wherein each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11} NR^{c11}C(O)R^{b11}, NR^{c11}C(O)OR^{a11}, and NR^{c11}C(O)NR^{c11}R^{d11}; and
each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl.

15. The compound of claim 1, or a pharmaceutically acceptable salt thereof, wherein each R^{1A} is independently selected from OR^{a11}, NR^{c11}C(O)R^{b11}, and NR^{c11}C(O)OR^{a11}; and
each R^{a11}, R^{b11}, and R^{c11} is independently selected from H, C₁₋₆ alkyl, and C₃₋₇ cycloalkyl.

16. The compound of claim 1, or a pharmaceutically acceptable salt thereof, wherein each R^{1A} is independently selected OH, —NHC(O)OCH₃, and —NHC(O) (cyclopropyl).

17. The compound of claim 1, or a pharmaceutically acceptable salt thereof, wherein R⁴ is selected from H and C₁₋₆ alkyl.

18. The compound of claim 1, or a pharmaceutically acceptable salt thereof, wherein R⁴ is C₁₋₆ alkyl.

19. The compound of claim 1, or a pharmaceutically acceptable salt thereof, wherein R⁴ is trideuteromethyl.

20. The compound of claim 1, or a pharmaceutically acceptable salt thereof, wherein R⁵ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

21. The compound of claim 1, or a pharmaceutically acceptable salt thereof, wherein R⁵ is H.

22. The compound of claim 1, or a pharmaceutically acceptable salt thereof, wherein Cy² is 5-10 membered heteroaryl, wherein the 5-10 membered heteroaryl of Cy² is optionally substituted with 1, 2, 3, or 4 independently selected R² substituents.

23. The compound of claim 1, or a pharmaceutically acceptable salt thereof, wherein Cy² is bicyclic 8-10 membered heteroaryl, wherein the bicyclic 8-10 membered heteroaryl of Cy² is optionally substituted with 1, 2, 3, or 4 independently selected R² substituents.

24. The compound of claim 1, or a pharmaceutically acceptable salt thereof, wherein Cy² is selected from quinolinyl and pyrazolo[4,3-b]pyridinyl, wherein the quinolinyl and pyrazolo[4,3-b]pyridinyl of Cy² are each optionally substituted with 1, 2, 3, or 4 independently selected R² substituents.

25. The compound of claim 1, or a pharmaceutically acceptable salt thereof, wherein each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents.

26. The compound of claim 1, or a pharmaceutically acceptable salt thereof, wherein each R² is independently selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents.

27. The compound of claim 1, or a pharmaceutically acceptable salt thereof, wherein each R² is independently selected from C₁₋₆ alkyl, C₁₋₆ haloalkyl, and 5-10 membered heteroaryl, wherein each C₁₋₆ alkyl and 5-10 membered heteroaryl of R² is optionally substituted with 1, 2, 3, or 4 independently selected R^{2A} substituents.

28. The compound of claim 1, or a pharmaceutically acceptable salt thereof, wherein each R² is independently selected from methyl, trifluoromethyl, and pyrazolyl, wherein each pyrazolyl of R² is optionally substituted with 1, 2, 3, or 4 independently selected R^{2A} substituents.

29. The compound of claim 1, or a pharmaceutically acceptable salt thereof, wherein each R^{2A} is independently selected from halo, C₁₋₆ alkyl, C₁₋₆ haloalkyl, C₂₋₆ alkenyl, and C₂₋₆ alkynyl.

30. The compound of claim 1, or a pharmaceutically acceptable salt thereof, wherein each R^{2A} is independently selected from C₁₋₆ alkyl and C₁₋₆ haloalkyl.

31. The compound of claim 1, or a pharmaceutically acceptable salt thereof, wherein each R^{2A} is trifluoromethyl.

32. The compound of claim 1, or a pharmaceutically acceptable salt thereof, wherein:
W is CR^{W} or C(R^{W})₂;
Y is CR^{Y} or C(R^{Y})₂;
each R^{W} is independently selected from H, oxo, and C₁₋₆ alkyl;
each R^{Y} is independently selected from H, oxo, and C₁₋₆ alkyl;
Ring A is C₃₋₁₀ cycloalkyl, wherein the C₃₋₁₀ cycloalkyl of Ring A is optionally substituted with 1, 2, 3, or 4 independently selected R¹ substituents;
each R¹ is independently selected from C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, NR^{c1}C(O)R^{b1}, NR^{c1}C(O)OR^{a1}, and NR^{c1}C(O)NR^{c1}R^{d1}, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}C(O)R^{b11}, NR^{c11}C(O)OR^{a11}, and NR^{c11}C(O)NR^{c11}R^{d11};
each R^{a11}, R^{b11}, R^{c11}, and R^{d11} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl;
Cy² is 5-10 membered heteroaryl, wherein the 5-10 membered heteroaryl of Cy² is optionally substituted with 1, 2, 3, or 4 independently selected R² substituents;
each R² is independently selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{2A} is independently selected from halo, C₁₋₆ alkyl, C₁₋₆ haloalkyl, C₂₋₆ alkenyl, and C₂₋₆ alkynyl;
R⁴ is selected from H and C₁₋₆ alkyl; and
R⁵ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

33. The compound of claim 1, or a pharmaceutically acceptable salt thereof, wherein:
W is C(R^{W})₂;
Y is C(R^{Y})₂;
each R^{W} is independently selected from H, oxo, and C₁₋₃ alkyl;
each R^{Y} is independently selected from H, oxo, and C₁₋₃ alkyl;
Ring A is C₃₋₇ cycloalkyl, wherein the C₃₋₇ cycloalkyl of Ring A is optionally substituted with 1, 2, 3, or 4 independently selected R¹ substituents;
each R¹ is independently selected from C₁₋₆ alkyl, C₁₋₆ haloalkyl, NR^{c1}C(O)R^{b1}, NR^{c1}C(O)OR^{a1}, and NR^{c1}C(O)NR^{c1}R^{d1}, wherein each C₁₋₆ alkyl of R¹ is optionally substituted with 1, 2, 3, or 4 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, NR^{c11}C(O)R^{b11}, and NR^{c11}C(O)OR^{a11};
each R^{a11}, R^{b11}, and R^{d11} is independently selected from H, C₁₋₆ alkyl, and C₃₋₇ cycloalkyl;
Cy² is bicyclic 8-10 membered heteroaryl, wherein the bicyclic 8-10 membered heteroaryl of Cy² is optionally substituted with 1, 2, 3, or 4 independently selected R² substituents;
each R² is independently selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, and C₂₋₆ alkynyl;
R⁴ is selected from H and C₁₋₆ alkyl; and
R⁵ is selected from H and C₁₋₆ alkyl.

34. The compound of claim 1, or a pharmaceutically acceptable salt thereof, wherein:
W is CH₂;
Y is CH₂;
Ring A is C₃₋₇ cycloalkyl, wherein the C₃₋₇ cycloalkyl of Ring A is optionally substituted with 1, 2, 3, or 4 independently selected R¹ substituents;
each R¹ is independently selected from C₁₋₆ alkyl and NR^{c1}C(O)NR^{c1}R^{d1}, wherein each C₁₋₆ alkyl of R¹ is optionally substituted with 1, 2, 3, or 4 independently selected R^{1A} substituents;
each R^{c1} and R^{d1} is independently selected from H, C₁₋₆ alkyl, C₁₋₆ haloalkyl, C₂₋₆ alkenyl, and C₂₋₆ alkynyl;
each R^{1A} is independently selected from OR^{a11}, NR^{c11}C(O)R^{b11}, and NR^{c11}C(O)OR^{a11};
each R^{a11}, R^{b11}, and R^{d11} is independently selected from H, C₁₋₆ alkyl, and C₃₋₇ cycloalkyl;
Cy² is bicyclic 8-10 membered heteroaryl, wherein the bicyclic 8-10 membered heteroaryl of Cy² is optionally substituted with 1, 2, 3, or 4 independently selected R² substituents;
each R² is independently selected from C₁₋₆ alkyl, C₁₋₆ haloalkyl, and 5-10 membered heteroaryl, wherein each C₁₋₆ alkyl and 5-10 membered heteroaryl of R² is optionally substituted with 1, 2, 3, or 4 independently selected R^{2A} substituents;
each R^{2A} is independently selected from C₁₋₆ alkyl and C₁₋₆ haloalkyl;
R⁴ is C₁₋₆ alkyl; and
R⁵ is H.

35. The compound of claim 1, wherein the compound of Formula I is a compound of Formula II: 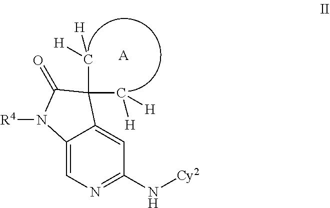
or a pharmaceutically acceptable salt thereof.

36. The compound of claim 1, wherein the compound of Formula I is a compound of Formula III: 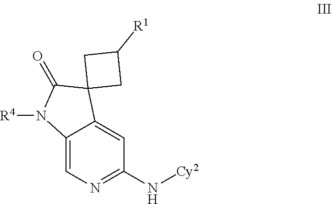
or a pharmaceutically acceptable salt thereof.

37. The compound of claim 1, wherein the compound of Formula I is a compound of Formula IIIa: 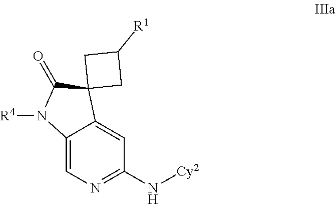
or a pharmaceutically acceptable salt thereof.

38. The compound of claim 1, wherein the compound of Formula I is a compound of Formula IV: 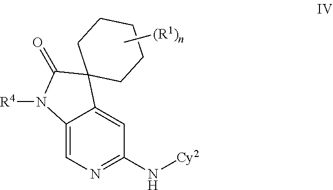
or a pharmaceutically acceptable salt thereof, wherein n is 0, 1, 2, 3, 4, 5, or 6.

39. The compound of claim 1, which is selected from:
1-ethyl-3-((1s,4s)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea;
1-ethyl-3-((1r,4r)-1′-(methyl-d₃)-2′-oxo-5′-((8-(trifluoromethyl)quinolin-2-yl)amino)-1′,2′-dihydrospiro[cyclohexane-1,3′-pyrrolo[2,3-c]pyridin]-4-yl)urea;
methyl (((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)carbamate;
N-(((1r,3r)-1′-(methyl-d₃)-2′-oxo-5′-((8-(trifluoromethyl)quinolin-2-yl)amino)-1′,2′-dihydrospiro[cyclobutane-1,3′-pyrrolo[2,3-c]pyridin]-3-yl)methyl)cyclopropanecarboxamide;
(1r,3r)-3-(hydroxymethyl)-1′-(methyl-d₃)-5′-((1-methyl-3-(1-(trifluoromethyl)-1H-pyrazol-4-yl)-1H-pyrazolo[4,3-b]pyridin-5-yl)amino)spiro[cyclobutane-1,3′-pyrrolo[2,3-c]pyridin]-2′(1′H)-one;
methyl (((1r,3r)-1′-(methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)carbamate; and
N-(((1r,3r)-1′-(methyl-d₃)-5′-((1-methyl-3-(1-(trifluoromethyl)-1H-pyrazol-4-yl)-1H-pyrazolo[4,3-b]pyridin-5-yl)amino)-2′-oxo-1′,2′-dihydrospiro[cyclobutane-1,3′-pyrrolo[2,3-c]pyridin]-3-yl)methyl)cyclopropanecarboxamide;
or a pharmaceutically acceptable salt thereof.

40. The compound of claim 1, or a pharmaceutically acceptable salt thereof, wherein the compound is deuterated.

41. A pharmaceutical composition, comprising a compound of claim 1, or a pharmaceutically acceptable salt thereof, and a pharmaceutically acceptable carrier.

42. A method of inhibiting an activity of the V617F variant of JAK2 kinase, comprising contacting the kinase with a compound of claim 1, or a pharmaceutically acceptable salt thereof.

43. A method of treating cancer in a patient in need thereof, the method comprising administering to the patient a therapeutically effective amount of a compound of claim 1, or a pharmaceutically acceptable salt thereof.

44. The method of claim 43, wherein the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, and brain cancer.

45. The method of claim 43, wherein the cancer is a hematological cancer.

46. The method of claim 43, wherein the cancer is selected from leukemia, lymphoma, multiple myeloma, chronic lymphocytic lymphoma, adult T cell leukemia, acute myeloid leukemia, B-cell lymphoma, cutaneous T-cell lymphoma, acute myelogenous leukemia, Hodgkin's or non-Hodgkin's lymphoma, a myeloproliferative neoplasm), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphoma, and Burkitt's lymphoma.

47. A method of treating a myeloproliferative disorder in a patient in need thereof, the method comprising administering to the patient a therapeutically effective amount of a compound of claim 1, or a pharmaceutically acceptable salt thereof.

48. The method of claim 47, wherein the myeloproliferative disorder is selected from polycythemia vera, essential thrombocythemia, myelofibrosis with myeloid metaplasia, primary myelofibrosis, post-essential thrombocythemia myelofibrosis, post polycythemia vera myelofibrosis, chronic myelogenous leukemia, chronic myelomonocytic leukemia, hypereosinophilic syndrome, and systemic mast cell disease.

49. A method of treating myelodysplastic syndrome in a patient in need thereof, the method comprising administering to the patient a therapeutically effective amount of a compound of claim 1, or a pharmaceutically acceptable salt thereof.
